(19) United States
(12) Patent Application Publication
(10) Pub. No.: US 2021/0000123 A1
(43) Pub. Date: Jan. 7, 2021

(54) ANTI-PARASITIC AGENTS

(71) Applicant: The University Court of the University of Glasgow, Strathclyde (GB)

(72) Inventors: Antony P. PAGE, Glasgow (GB); Marie-Anne FELIX, Paris (FR)

(21) Appl. No.: 16/898,683

(22) Filed: Jun. 11, 2020

(30) Foreign Application Priority Data
Jun. 11, 2019 (GB) 1908318.7

(51) Int. Cl. A01N 63/20; A61K 35/74

(52) U.S. Cl. CPC A01N 63/20; A61K 35/74

(57) ABSTRACT

The invention relates to anti-parasitic agents, and particularly, although not exclusively, to the use of Chryseobacterium nematophagum for the control of nematode parasites.

### CROSS-REFERENCE TO RELATED APPLICATIONS

The present application claims priority to U.K. Patent Application No. 1908318.7, entitled Anti-Parasitic Agents, filed 11 Jun. 2019.

### FIELD OF THE INVENTION

The present invention relates to anti-parasitic agents, and particularly, although not exclusively, to the use of Chryseobacterium nematophagum for the control of nematode parasites.

### BACKGROUND

Parasitic nematodes inflict a major burden on public health and on the farming industry worldwide. It is estimated that more than one billion people are suffering from soil-transmitted nematode infestations, such as hookworm infection, Ascariasis and Trichuriasis. These parasites cause significant lifelong morbidity [1]. The veterinary impact of disease caused by nematodes is enormous, with an estimated annual economic loss of $2 billion to livestock production in North America alone [2]. Moreover, the losses in crop yields caused by plant parasitic nematodes are estimated to be as much as $125 billion per year [3].

Several classes of highly effective anthelmintic molecules were introduced, first for veterinary purposes, such as the benzimidazoles (e.g. albendazole) and the imidazothiazoles (e.g. levamisole) in the 1960s, the macrocyclic lactones (e.g. ivermectin and moxidectin) in the 1980s, and the novel amino-acetonitrile derivative monepantel in 2009 [4]. However, drug resistance has arisen unexpectedly rapidly, mainly in South Africa, USA and Australia with for example, the first cases of resistance to benzimidazoles reported within five years of drug release [5]. Resistance to ivermectin and other macrocyclic lactones has now also been reported in numerous countries with intensive farming activities. The problem of drug resistance strikes the global sheep industry particularly hard, with resistance prevalence often exceeding 50% of all infections [6]. Moreover, anthelmintic resistance is irreversible [7]. It is predicted that the high levels of resistance currently observed in veterinary parasites will ultimately develop in the soil-transmitted nematodes of humans [6]. Despite decades of research, only two species-specific vaccines with limited application are currently available [8]. With the above control limitations, there is a pressing need to develop new methods of nematode control, particularly for the ubiquitous Trichostrongylid parasites of livestock.

The Trichostrongyles have obligate environmental developmental stages (egg-L3) and while grazing management methods can to some extent limit parasite exposure, an important unexploited means of control is the application of natural predators of these free-living stages. One such method is the nematode-trapping fungus Duddington flagrans that has been shown to reduce the pasture levels of infective larvae [9]. In the case of plant parasitic nematodes, biocontrol through the application of species-specific bacterial pathogens, such as Pasteuria penetrans, is well established [10] and is now commercially available. A current unmet need is to discover and develop new biocontrol measures that will reduce the larval infection of pasture with Trichostrongyles to a level that avoids both clinical and sub-clinical disease in grazing livestock. Such control measures will help curtail resistance thereby preserving the available anthelmintics for the treatment of diseased animals.

The present invention has been devised in light of the above considerations.

### SUMMARY OF THE INVENTION

The inventors have surprisingly found that the bacterium Chryseobacterium nematophagum (C. nematophagum) has pathogenic activity against a wide range of nematode species, including parasitic species.

Thus, in its broadest form, the invention relates to the use of Chryseobacterium nematophagum as a parasite control agent, specifically for control of parasitic nematodes, i.e. nematodes which are parasitic for human, veterinary or plant species. The invention also relates to anti-parasitic compositions comprising Chryseobacterium nematophagum and their use.

Thus the invention provides a method of reducing parasitic nematode load in an environment, comprising treating the environment with a composition comprising Chryseobacterium nematophagum.

A large proportion of nematodes which are parasitic for mammals, including humans, are transmitted via the faeces of an infected mammalian host. Typically eggs contained within faeces will hatch after several days under appropriate environmental conditions. The resulting larvae develop into an infectious form which infects a new host on ingestion by that new host. The larvae continue their development into adults within the host's digestive tract, often the small intestine.

C. nematophagum is capable of killing multiple life stages of parasitic nematodes, including larval and adult forms. Thus, by treating an environment with C. nematophagum, the load of infectious nematodes within that environment will be reduced, leading to reduced infection of new hosts, and so lower rates of transmission.

The environment to be treated may be a habitat for domesticated mammals.

The domesticated mammals may include livestock, equine species (e.g. horses and donkeys), and domestic (e.g. companion) animals including cats and dogs.

Livestock include ruminants, which may be bovine (e.g. cows or cattle), caprine (e.g. goats), or ovine (e.g. sheep), as well as monogastric livestock (e.g. pigs).

The environment may comprise pasture or rangeland and buildings or other structures therein, such as animal sheds, barns, kennels, etc., which may house, or be intended to house, the domesticated mammals. Thus, compositions comprising C. nematophagum may be employed in an environment which already contains the relevant animals (e.g. to control parasitic nematode load within an existing animal population), or in an environment which does not yet contain animals (e.g. to reduce the load of parasitic nematodes already present in that environment, or to minimise transmission of parasitic nematodes once animals are introduced).

Additionally or alternatively, the environment may be a human residential or leisure environment, including buildings or other structures therein. The environment may contain humans and domesticated mammals (e.g. livestock, horses and/or companion animals), potentially in relatively close proximity. The environment may contain latrines, or other areas of human or animal defecation, which are liable to be potential sites or sources of nematode infection. The environment may contain buildings or other structures commonly inhabited by humans and/or animals, including human residences, animal sheds, barns, kennels, etc.

The methods may comprise contacting soil or vegetation within the environment with the composition.

The methods may comprise spraying the composition within the environment, e.g. spraying the composition onto soil or vegetation within the environment. However, the composition may be contacted with (e.g. sprayed onto) any suitable object or surface in the environment which is likely to come into contact with parasitic nematodes, whether adult or larval.

In such methods, the parasitic nematodes are typically mammalian parasites.

The methods of the invention are also applicable to controlling nematodes which are parasitic for plants, especially cultivated plants.

Thus, in some embodiments, the environment is a habitat for cultivated plants.

For example, the environment may comprise arable or cultivated land, and buildings or other structures therein.

The cultivated plants may be crop plants. For example, they may be fruit, vegetable, cereal or tree crops. Specific examples may include soy bean, potato, tomato, sugarcane, coffee, banana, maize, legumes, citrus, coconut, avocado, sugarbeet, grasses, rice, nuts, mushrooms, beans, onion, garlic, peas, celery, strawberries, beetroot, vegetable marrow, pumpkin, rhubarb, ornamental bulbs, oats and rye, grapevine, and coniferous trees such as pine trees.

As with methods intended to target animal parasites, the methods may comprise contacting soil or vegetation within the environment with the composition.

The methods may comprise directly contacting the cultivated plants with the composition.

Alternatively it may comprise contacting vegetation other than the cultivated plants with the composition.

The methods may comprise spraying the composition within the environment, e.g. spraying the composition onto soil or vegetation (which may be the cultivated plants or vegetation other than the cultivated plants).

The term “habitat” intended here to include any environment containing (or intended to contain) the relevant animals or plants, whether permanently or temporarily. Thus, in the context of animals, it may include pastureland, rangeland, enclosures, farm yards, buildings such as barns, animal sheds, kennels (and other animal accommodation, whether permanent or temporary), transit containers, vehicles, etc. In the context of plants, it may include arable land, cultivated land, ploughed land awaiting planting or sowing, greenhouses, glasshouses and other covered growing areas, plant containers, etc. . . . . Agricultural land in general can be a habitat for either or both of the relevant plants and animals.

Compositions comprising C. nematophagum may also be administered directly to animals.

Such methods are encompassed within the scope of the invention.

Thus in a further aspect the invention provides a method of reducing nematode load in an animal, for example in the alimentary canal of an animal, comprising administering to the animal a composition comprising Chryseobacterium nematophagum.

In such methods, the composition will be formulated so that C. nematophagum is active in the alimentary canal of the animal, e.g. in the small intestine.

In general, C. nematophagum is believed to have low viability at mammalian physiological temperature (37° C.) so may not have significant activity against parasites in the alimentary canal.

However, C. nematophagum may be formulated so as to pass through the alimentary canal and be egested as part of the animal's faeces. The bacterium will then be available and active against larvae hatching from nematode eggs within the animal's faeces. Such uses may not reduce the parasite burden of the animal to which the C. nematophagum is administered, but may reduce transmission of parasites between animals in a given population, e.g. within the same environment.

Thus the invention provides a method of reducing nematode transmission between animals (e.g. within an animal population) comprising administering to an animal a composition comprising Chryseobacterium nematophagum.

Typically the animals are mammals, e.g. domesticated mammals.

As in other aspects of the invention, the domesticated mammals may include livestock, horses and domestic (e.g. companion) animals such as cats, dogs, etc.

Livestock include ruminants, which may be bovine (e.g. cows or cattle), caprine (e.g. goats), or ovine (e.g. sheep) or monogastric (e.g. pigs).

Administration may be by feeding the composition to the animal.

For example, the composition may be administered in admixture with a foodstuff.

It will also be clear that C. nematophagum can be applied directly to plants. Thus the invention further provides a method of reducing parasitic nematode load of a plant, or of inhibiting colonisation of a plant by nematode parasites, the method comprising contacting the plant with a composition comprising Chryseobacterium nematophagum. The composition may be applied to any part of the plant, including leaves, stems or roots, particularly the roots. The method may comprise contacting the plant with the composition prior to planting, e.g. contacting the roots of the plant with the composition prior to planting.

In all aspects of the invention, the composition may comprise lyophilised (i.e. freeze-dried)C. nematophagum. Lyophilised C. nematophagum may be coated or encapsulated to avoid inadvertent rehydration. This may be particularly appropriate in compositions intended for administration (e.g. feeding) to animals, to help the bacteria survive passage through the digestive tract and pass out in the animal's faeces.

In some aspects of the invention, however, it may be possible simply to employ an active suspension, e.g. aqueous suspension, of C. nematophagum (i.e. hydrated and metabolically active), such as a crude fermentation broth or a more purified suspension of the bacteria.

The composition may comprise at least one excipient or carrier, depending on its intended use. The skilled person is capable of identifying suitable excipients for a given formulation.

The invention further provides a composition comprising lyophilised C. nematophagum, optionally in combination with a suitable carrier or excipient

The lyophilised C. nematophagum may be coated or encapsulated.

The composition may be admixed with an animal feedstuff. Thus the invention provides an animal feedstuff in admixture with a composition comprising lyophilised C. nematophagum, optionally wherein the lyophilised C. nematophagum is coated or encapsulated.

The invention further provides an article of manufacture comprising a composition comprising C. nematophagum, and adapted to deliver said composition to an environment or target site by spraying. The composition is typically a suspension of C. nematophagum, e.g. an aqueous suspension of C. nematophagum. The composition may comprise a fermentation broth comprising C. nematophagum. Alternatively the composition may comprise lyophilised C. nematophagum, which may optionally be coated or encapsulated. Thus the article of manufacture may be a manually operated spray device, such as a hand-held sprayer or backpack-type spray device, or a larger device such as an agricultural trailer or a self-propelled crop-row sprayer. Alternatively the article of manufacture may be a reservoir of the composition, adapted for connection to a spray nozzle of any suitable spray device.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures.

FIG. 1. Phylogenetic analysis of C. nematophagum isolates

A maximum likelihood tree was constructed using the 16S rRNA gene sequences of a number of Chryseobacterium species, including C. nematophagum (JUb129 and JUb275). The tree was rooted using a Reimerella anatipestifer 16S sequence. Shading indicates the bacterial species with the nematode-killing phenotype (C. nematophagum strains JUb129 and JUb257), and those experimentally demonstrated not to display the same phenotype (C. indoltheticum, C. shigense, C. indologenes, C. gallinarum and C. contaminans). Nodes with >80% bootstrap support are indicated with an asterisk.

FIG. 2. Time-course of C. elegans killing by C. nematophagum

Timecourse of 241 L1 C. elegans survival (% alive) in presence of OP50 (gray), compared to 193 L1 C. elegans in presence of C. nematophagum (black).

FIG. 3. Chryseobacterium nematophagum degrade the collagenous matrix of Caenorhabditis elegans.

COL-12 TY tagged C. elegans strains (IA132) were incubated with C. nematophagum JUb129 for 24 hrs (1) 48 hrs (3) and with OP50 alone control for 48 hrs (2). IA132 was incubated for 48 hrs with non-pathogenic Chryseobacterium indologenes (4). IA132 adults were pre-cleared of OP50-1 by washing in M9 buffer and culturing on non-seeded plates for 4 hours then incubated for 48 hrs with C. nematophagum JUb129 (5) or JUb275 (6). Twenty adult worms per treatment were extracted and Western blotted and probed with anti-TY tag (upper) then re-probed anti-β-actin (lower) antibodies.

FIG. 4. Genomic loci of candidate genes and domain structure of collagenase, chitinase and astacin enzymes

A. Genomic location of nematode killing phenotype-associated candidate genes as identified in the top-ranking hierarchical orthologous groups. B. The domain architecture of the C. nematophagum-specific collagenase, chitinase and astacin proteins is illustrated with the IDs for the orthologous sequences in JUb129 and JUb275 shown. The scale represents the number of amino acid residues and the legend shows the relevant protein motif database.

FIG. 5. Diagnostic PCR to identify C. nematophagum.

Panel 1: PCR reaction with primers CnemF1 and CnemR1 amplifies a 129 bp fragment from C. nematophagum JUb275 (lane 1) and JUb129 (lane 2) but not C. indologenes (lane 3) or E. coli (lane 4).

Panel 2: PCR reaction with primers CnemF1 and CnemR2 amplifies a 394 bp fragment from C. nematophagum JUb275 (lane 5) and JUb129 (lane 6) but not C. indologenes (lane 7) or E. coli (lane 8). (“M”=100 bp DNA markers (New England Biolabs).)

FIG. 6. Alignment of JUb129 and JUb275 with available Chryseobacterium 16S ribosomal RNA sequences, showing location of diagnostic primers.

Primer positions are indicated in bold and underlined.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Parasitic Nematodes and their Hosts

Nematodes or roundworms constitute the phylum Nematoda.

Nematodes are known to parasitise humans, animals (including livestock and domestic animals), plants, and insects. Many are bacterivorous, or have one or more life cycle stages (e.g. larval stages, such as L1, L2 and/or L3 larval stages) which are bacterivorous. Without wishing to be bound by theory, it is believed that the ingestion of C. nematophagum by nematodes may be significant in the killing of nematodes by C. nematophagum. Thus the nematodes targeted by the methods and compositions of the present invention may be bacterivorous, or may have at least one bacterivorous life stage. Even where the nematodes are not typically bacterivorous, they may nevertheless ingest bacteria at certain stages of their life cycle, such as during invasion of a host. This may particularly be the case with plant parasites, especially those which invade plant roots.

Nematodes capable of parasitising humans include Ascarididae (including Ascaris lumbricoides, capable of causing Ascariasis), Filarioidea, hookworms (e.g. of the genus Ancylostoma, such as Ancylostoma duodenale, and Necator, such as Necator americanus), Strongyloides including pinworms (e.g. Strongyloides stercoralis, capable of causing strongyloidiasis) and whipworms (Trichuris trichiura).

Many nematodes considered as veterinary parasites (e.g. capable of infecting livestock and domestic animals) belong to the Order Strongylida. Nematodes of this Order have environmental pre-infective larval stages L1, L2 and L3 which are bacterivorous and hence are capable of ingesting C. nematophagum.

Certain of these belong to the family Trichostrongylidae, including:

Haemonchus contortus, affecting ruminants (especially sheep and goats);
Trichostrongylus vitrinus, affecting ruminants (especially sheep and goats);
Teladorsagia circumcinta, affecting ruminants (especially sheep and goats);
Ostertagia ostertagi, affecting ruminants, particularly cattle (causing ostertagiosis), but also sheep, goats and wild ruminants, and also horses;
Dictyocaulus viviparus (lungworm of ruminants, especially cattle);
Dictyocaulus filaria (lungworm of ruminants, especially sheep and goats);
Dictyocaulus amfieldi (lungworm of equine species, especially horses and donkeys);
Cooperia curtecei (affecting ruminants, especially sheep and goats);
Cooperia oncophera (affecting ruminants, especially cattle).

Others belong to the family Strongylidae, including:

Cyathastomins, affecting horses (potentially causing parasitic colitis, acute diarrhoea and colic);
Strongylus spp. (affecting equine species such as horses and donkeys);
Chabertia ovina (affecting ruminants, especially sheep and goats);
Oesophagostomum spp. (affecting ruminants, especially sheep, goats and cattle, and monogastric livestock, especially pig);
Stephanurus dentatus (affecting monogastric livestock, especially pig);
Ancylostoma spp (e.g. A. caninum, A. tubaeforme, A. braziliense; affecting dogs and cats);
Uncinaria stenocephala (affecting dogs and cats);
Bunostomum spp (affecting ruminants, especially sheep and cattle);
Metastrongylus spp. (affecting ruminants, especially sheep and goats, and monogastric livestock, especially pigs).

Still others belong to the family Rhadditoidea, including:

Strongyloides spp (affecting equine species, e.g. horses and donkeys; ruminants, especially cattle and sheep; cats and dogs).

There are over 4000 species of plant parasitic nematodes, estimated to cause over $80 billion worth of crop damage per year. The majority fall within one of three main groups: the root knot nematodes (such as Meloidygyne spp.), the cyst nematodes (such as Globodera and Heterodera spp.), and the root lesion nematodes (such as Pratylenchus spp.). These diverse nematodes are typically found in soil and have specialized mouthparts adapted for infecting and feeding on root structures.

Thus, important nematode parasites of plants include:

Meloidogynidae, including root knot nematodes (RKN) of the genus Meloidogyne (e.g. M. javanica, M. arenaria, M. incognita, M. hapla. Commonly affected hosts include cassava, potato, soy bean, tea, cereals, grapevines and other fruit and vegetable crops.

Potato cyst nematodes (PCN) of the genus Globodera (e.g. Globodera achilleae, Globodera artemisiae, Globodera chaubattia, Globodera effingtonae, Globodera hypolysi, Globodera leptonepia, Globodera millefolii, Globodera mirabilis, Globodera pallida, Globodera pseudorostochiensis, Globodera rostochiensis, Globodera tabacum, Globodera zelandica). As their name suggests, PCN primarily affect potato, but also tobacco crops.

Soybean cyst nematodes (SONS) of the genus Heterodera, e.g. Heterodera glycines. SON primarily affect soybean crops.

Cereal cyst nematodes (CCNs) of the genus Heterodera, e.g. Heterodera avenae and H. filipjevi. CONs primarily affect cereal crops including ryegrass, barley, oats and wheat.

Root lesion nematodes of the genus Pratylenchus (e.g. Pratylenchus alleni, Pratylenchus brachyurus, Pratylenchus coffeae, Pratylenchus crenatus, Pratylenchus dulscus, Pratylenchus fallax, Pratylenchus flakkensis, Pratylenchus goodeyi, Pratylenchus hexincisus, Pratylenchus loosi, Pratylenchus minutus, Pratylenchus mulchandi, Pratylenchus musicola, Pratylenchus neglectus, Pratylenchus penetrans, Pratylenchus pratensis, Pratylenchus reniformia, Pratylenchus scribneri, Pratylenchus thomei, Pratylenchus vulnus, Pratylenchus zeae). Root lesion nematodes affect many crops including cereals (especially wheat), sugarcane, coffee, banana, maize, legumes, canola, chickpea, potato, and many vegetables and fruit trees.

The burrowing nematode Radopholus similis, which primarily affects banana, citrus crops and pepper, as well as coconut, avocado, coffee, sugarcane, and grasses.

Ditylenchus spp. including Ditylenchus angustus (rice stem nematode, affecting primarily rice), D. destructor (potato rot nematode, affecting primarily potato), D. africanus (affecting primarily peanuts and groundnuts), D. myceliophagus (affecting primarily mushroom crops, such as Agaricus bisporus), D. gigas (affecting primarily beans), and D. dipsaci (affecting primarily infects onion and garlic, but also many other crop species including peas, celery, strawberries, beetroot, vegetable marrow, pumpkin, rhubarb, ornamental bulbs (hyacinth, narcissus and tulip), oats and rye).

The pine wilt nematode Bursaphelenchus xylophilus (primarily affecting timber crops, including coniferous trees, especially pine trees).

The reniform nematode Rotylenchulus reniformis (affecting many vegetable, fruit, fibre and ornamental crops including citrus fruits and coffee).

Xiphinema index, affecting primarily grapevine (Vitis spp.) crops.

Nacobbus aberrans, affecting vegetable crops including tomato, beans, chilli pepper and sugarbeet.

Aphelenchoides besseyi, affecting primarily rice and strawberry, but also numerous other monocotyledonous and dicotyledonous species.

Chryseobacterium nematophagum

Bacteria of the genus Chryseobacterium are Gram-negative, rod-shaped, chemoorganotrophic, catalase positive and oxidase positive. Colonies are typically mucoid and have a characteristic golden colour due to production of a flexirubin-type pigment.

In API 20E and API 20NE tests, Chryseobacterium nematophagum are positive for gelatin, esculin and N-acetylglucosamine, and give the closest identification to Chryseobacterium indologenes.

The strain JUb275 possesses 6 identical copies of the 16S small subunit (SSU) gene, each having the sequence:

(SEQ ID NO: 1)
GATGAACGCTAGCGGGAGGCCTAACACATGCAAGCCGAGCGGTATGATTC
TTTCGGGAATCAGAGAGCGGCGTACGGGTGCGGAACACGTGTGCAACCTG
CCTTTATCTGGGGGATAGCCTTTCGAAAGGAAGATTAATACCCCATAATA
TATTGATTGGCATCGATTGATATTGAAAACTCCGGTGGATAAAGATGGGC
ACGCGCAAGATTAGATAGTTGGTGAGGTAACGGCTCACCAAGTCGATGAT
CTTTAGGGGGCCTGAGAGGGTGATCCCCCACACTGGTACTGAGACACGGA
CCAGACTCCTACGGGAGGCAGCAGTGAGGAATATTGGACAATGGGTGCAA
GCCTGATCCAGCCATCCCGCGTGAAGGACGACGGCCCTATGGGTTGTAAA
CTTCTTTTGTACAGGGATAAACCTTTCCACGTGTGGGAAGCTGAAGGTAC
TGTACGAATAAGCACCGGCTAACTCCGTGCCAGCAGCCGCGGTAATACGG
AGGGTGCAAGCGTTATCCGGATTTATTGGGTTTAAAGGGTCCGTAGGCGG
ATTTGTAAGTCAGTGGTGAAATCCTACAGCTTAACTGTAGAACTGCCATT
GATACTGCAAGTCTTGAGTGTAGTTGAAGTAGCTGGAATAAGTAGTGTAG
CGGTGAAATGCATAGATATTACTTAGAACACCAATTGCGAAGGCAGGTTA
CTAAGTTACAACTGACGCTGATGGACGAAAGCGTGGGGAGCGAACAGGAT
TAGATACCCTGGTAGTCCACGCTGTAAACGATGCTAACTCGTTTTTGGGT
TTTCGGATTCAGAGACTAAGCGAAAGTGATAAGTTAGCCACCTGGGGAGT
ACGAACGCAAGTTTGAAACTCAAAGGAATTGACGGGGGCCCGCACAAGCG
GTGGATTATGTGGTTTAATTCGATGATACGCGAGGAACCTTACCAAGGCT
TAAATGGGAATTGACAGATTTAGAAATAGATCCTCCTTCGGGCAATTTTC
AAGGTGCTGCATGGTTGTCGTCAGCTCGTGCCGTGAGGTGTTAGGTTAAG
TCCTGCAACGAGCGCAACCCCTGTCACTAGTTGCCATCATTAAGTTGGGG
ACTCTAGTGAGACTGCCTACGCAAGTAGAGAGGAAGGTGGGGATGACGTC
AAATCATCACGGCCCTTACGCCTTGGGCCACACACGTAATACAATGGCCG
GTACAGAGGGCAGCTACACAGCGATGTGATGCAAATCTCGAAAGCCGGTC
TCAGTTCGGATTGGAGTCTGCAACTCGACTCTATGAAGCTGGAATCGCTA
GTAATCGCGCATCAGCCATGGCGCGGTGAATACGTTCCCGGGCCTTGTAC
ACACCGCCCGTCAAGCCATGGAAGTCTGGGGTACCTGAAGTCGGTGACCG
TAAAAGGAGCTGCCTAGGGTAAAACAG

The strain JUb129 possesses 5 identical copies of the 16S small subunit (SSU) gene, each having the sequence:

(SEQ ID NO: 2)
GATGAACGCTAGCGGGAGGCCTAACACATGCAAGCCGAGCGGTATGATTC
TTTCGGGAATCAGAGAGCGGCGTACGGGTGCGGAACACGTGTGCAACCTG
CCTTTATCTGGGGGATAGCCTTTCGAAAGGAAGATTAATACCCCATAATA
TATTGATTGGCATCGATTAATATTGAAAACTCCGGTGGATAAAGATGGGC
ACGCGCAAGATTAGATAGTTGGTGAGGTAACGGCTCACCAAGTCCATGAT
CTTTAGGGGGCCTGAGAGGGTGATCCCCCACACTGGTACTGAGACACGGA
CCAGACTCCTACGGGAGGCAGCAGTGAGGAATATTGGACAATGGGTGAAA
GCCTGATCCAGCCATCCCGCGTGAAGGACGACGGCCCTATGGGTTGTAAA
CTTCTTTTGTACAGGGATAAACCTTTCCACGTGTGGGAAGCTGAAGGTAC
TGTACGAATAAGCACCGGCTAACTCCGTGCCAGCAGCCGCGGTAATACGG
AGGGTGCAAGCGTTATCCGGATTTATTGGGTTTAAAGGGTCCGTAGGCGG
ATTTGTAAGTCAGTGGTGAAATCCTACAGCTTAACTGTAGAACTGCCATT
GATACTGCAAGTCTTGAGTGTAGTTGAAGTAGCTGGAATAAGTAGTGTAG
CGGTGAAATGCATAGATATTACTTAGAACACCAATTGCGAAGGCAGGTTA
CTAAGTTACAACTGACGCTGATGGACGAAAGCGTGGGGAGCGAACAGGAT
TAGATACCCTGGTAGTCCACGCTGTAAACGATGCTAACTCGTTTTTGGGT
TTTCGGATTCAGAGACTAAGCGAAAGTGATAAGTTAGCCACCTGGGGAGT
ACGAACGCAAGTTTGAAACTCAAAGGAATTGACGGGGGCCCGCACAAGCG
GTGGATTATGTGGTTTAATTCGATGATACGCGAGGAACCTTACCAAGGCT
TAAATGGGAATTGACAGATTTAGAAATAGATCCTCCTTCGGGCAATTTTC
AAGGTGCTGCATGGTTGTCGTCAGCTCGTGCCGTGAGGTGTTAGGTTAAG
TCCTGCAACGAGCGCAACCCCTGTCACTAGTTGCCATCATTAAGTTGGGG
ACTCTAGTGAGACTGCCTACGCAAGTAGAGAGGAAGGTGGGGATGACGTC
AAATCATCACGGCCCTTACGCCTTGGGCCACACACGTAATACAATGGCCG
GTACAGAGGGCAGCTACACAGCGATGTGATGCAAATCTCGAAAGCCGGTC
TCAGTTCGGATTGGAGTCTGCAACTCGACTCTATGAAGCTGGAATCGCTA
GTAATCGCGCATCAGCCATGGCGCGGTGAATACGTTCCCGGGCCTTGTAC
ACACCGCCCGTCAAGCCATGGAAGTCTGGGGTACCTGAAGTCGGTGACCG
TAAAAGGAGCTGCCTAGGGTAAAACAG

plus a further copy having one single-nucleotide polymorphism:

(SEQ ID NO: 3)
GATGAACGCTAGCGGGAGGCCTAACACATGCAAGCCGAGCGGTATGATTC
TTTCGGGAATCAGAGAGCGGCGTACGGGTGCGGAACACGTGTGCAACCTG
CCTTTATCTGGGGGATAGCCTTTCGAAAGGAAGATTAATACCCCATAATA
TATTGATTGGCATCGATTRATATTGAAAACTCCGGTGGATAAAGATGGGC
ACGCGCAAGATTAGATAGTTGGTGAGGTAACGGCTCACCAAGTCCATGAT
CTTTAGGGGGCCTGAGAGGGTGATCCCCCACACTGGTACTGAGACACGGA
CCAGACTCCTACGGGAGGCAGCAGTGAGGAATATTGGACAATGGGTGAAA
GCCTGATCCAGCCATCCCGCGTGAAGGACGACGGCCCTATGGGTTGTAAA
CTTCTTTTGTACAGGGATAAACCTTTCCACGTGTGGGAAGCTGAAGGTAC
TGTACGAATAAGCACCGGCTAACTCCGTGCCAGCAGCCGCGGTAATACGG
AGGGTGCAAGCGTTATCCGGATTTATTGGGTTTAAAGGGTCCGTAGGCGG
ATTTGTAAGTCAGTGGTGAAATCCTACAGCTTAACTGTAGAACTGCCATT
GATACTGCAAGTCTTGAGTGTAGTTGAAGTAGCTGGAATAAGTAGTGTAG
CGGTGAAATGCATAGATATTACTTAGAACACCAATTGCGAAGGCAGGTTA
CTAAGTTACAACTGACGCTGATGGACGAAAGCGTGGGGAGCGAACAGGAT
TAGATACCCTGGTAGTCCACGCTGTAAACGATGCTAACTCGTTTTTGGGT
TTTCGGATTCAGAGACTAAGCGAAAGTGATAAGTTAGCCACCTGGGGAGT
ACGAACGCAAGTTTGAAACTCAAAGGAATTGACGGGGGCCCGCACAAGCG
GTGGATTATGTGGTTTAATTCGATGATACGCGAGGAACCTTACCAAGGCT
TAAATGGGAATTGACAGATTTAGAAATAGATCCTCCTTCGGGCAATTTTC
AAGGTGCTGCATGGTTGTCGTCAGCTCGTGCCGTGAGGTGTTAGGTTAAG
TCCTGCAACGAGCGCAACCCCTGTCACTAGTTGCCATCATTAAGTTGGGG
ACTCTAGTGAGACTGCCTACGCAAGTAGAGAGGAAGGTGGGGATGACGTC
AAATCATCACGGCCCTTACGCCTTGGGCCACACACGTAATACAATGGCCG
GTACAGAGGGCAGCTACACAGCGATGTGATGCAAATCTCGAAAGCCGGTC
TCAGTTCGGATTGGAGTCTGCAACTCGACTCTATGAAGCTGGAATCGCTA
GTAATCGCGCATCAGCCATGGCGCGGTGAATACGTTCCCGGGCCTTGTAC
ACACCGCCCGTCAAGCCATGGAAGTCTGGGGTACCTGAAGTCGGTGACCG
TAAAAGGAGCTGCCTAGGGTAAAACAG

All copies of the 16S SSU RNA gene are predicted to be 1427 nucleotides in length.

The JUb129 consensus sequence and the JUb275 sequence differ at only three positions, providing the following C. nematophagum consensus sequence:

(SEQ ID NO: 4)
GATGAACGCTAGCGGGAGGCCTAACACATGCAAGCCGAGCGGTATGATTC
TTTCGGGAATCAGAGAGCGGCGTACGGGTGCGGAACACGTGTGCAACCTG
CCTTTATCTGGGGGATAGCCTTTCGAAAGGAAGATTAATACCCCATAATA
TATTGATTGGCATCGATTRATATTGAAAACTCCGGTGGATAAAGATGGGC
ACGCGCAAGATTAGATAGTTGGTGAGGTAACGGCTCACCAAGTCSATGAT
CTTTAGGGGGCCTGAGAGGGTGATCCCCCACACTGGTACTGAGACACGGA
CCAGACTCCTACGGGAGGCAGCAGTGAGGAATATTGGACAATGGGTGMAA
GCCTGATCCAGCCATCCCGCGTGAAGGACGACGGCCCTATGGGTTGTAAA
CTTCTTTTGTACAGGGATAAACCTTTCCACGTGTGGGAAGCTGAAGGTAC
TGTACGAATAAGCACCGGCTAACTCCGTGCCAGCAGCCGCGGTAATACGG
AGGGTGCAAGCGTTATCCGGATTTATTGGGTTTAAAGGGTCCGTAGGCGG
ATTTGTAAGTCAGTGGTGAAATCCTACAGCTTAACTGTAGAACTGCCATT
GATACTGCAAGTCTTGAGTGTAGTTGAAGTAGCTGGAATAAGTAGTGTAG
CGGTGAAATGCATAGATATTACTTAGAACACCAATTGCGAAGGCAGGTTA
CTAAGTTACAACTGACGCTGATGGACGAAAGCGTGGGGAGCGAACAGGAT
TAGATACCCTGGTAGTCCACGCTGTAAACGATGCTAACTCGTTTTTGGGT
TTTCGGATTCAGAGACTAAGCGAAAGTGATAAGTTAGCCACCTGGGGAGT
ACGAACGCAAGTTTGAAACTCAAAGGAATTGACGGGGGCCCGCACAAGCG
GTGGATTATGTGGTTTAATTCGATGATACGCGAGGAACCTTACCAAGGCT
TAAATGGGAATTGACAGATTTAGAAATAGATCCTCCTTCGGGCAATTTTC
AAGGTGCTGCATGGTTGTCGTCAGCTCGTGCCGTGAGGTGTTAGGTTAAG
TCCTGCAACGAGCGCAACCCCTGTCACTAGTTGCCATCATTAAGTTGGGG
ACTCTAGTGAGACTGCCTACGCAAGTAGAGAGGAAGGTGGGGATGACGTC
AAATCATCACGGCCCTTACGCCTTGGGCCACACACGTAATACAATGGCCG
GTACAGAGGGCAGCTACACAGCGATGTGATGCAAATCTCGAAAGCCGGTC
TCAGTTCGGATTGGAGTCTGCAACTCGACTCTATGAAGCTGGAATCGCTA
GTAATCGCGCATCAGCCATGGCGCGGTGAATACGTTCCCGGGCCTTGTAC
ACACCGCCCGTCAAGCCATGGAAGTCTGGGGTACCTGAAGTCGGTGACCG
TAAAAGGAGCTGCCTAGGGTAAAACAG

where:

### R=A or G

### S=C or G

### M=A or C

The JUb129 consensus sequence has 99.8% identity to the JUb275 sequence and 96.2% identity to the 16S SSU RNA from C. indologenes strain B7 (HQ259684.1). The C. nematophagum consensus sequence of SEQ ID NO: 4 has 95.5% identity to the 16S SSU RNA from C. indologenes strain B7 (HQ259684.1).

A Chryseobacterium nematophagum may comprise a 16S SSU rRNA sequence that satisfies the consensus sequence of SEQ ID NO: 4, or has at least 98% identity to the consensus sequence of SEQ ID NO: 4, e.g. at least 98.5%, 99.0% or 99.5% identity to SEQ ID NO: 4.

It may contain multiple copies of 16S SSU rRNA genes, e.g. 6 copies, one or more of which satisfies the consensus sequence of SEQ ID NO: 4, or has at least 98% identity to the consensus sequence of SEQ ID NO: 4. For example, all copies of the 16S SSU rRNA gene may satisfy the consensus sequence of SEQ ID NO: 4, or have at least 98% identity to the consensus sequence of SEQ ID NO: 4, e.g. at least 98.5%, 99.0% or 99.5% identity to SEQ ID NO: 4.

Where a Chryseobacterium nematophagum comprises 16S SSU rRNA genes having two or more different sequences, their consensus sequence may satisfy the consensus sequence of SEQ ID NO: 4, or have at least 98% identity to the consensus sequence of SEQ ID NO: 4, e.g. at least 98.5%, 99.0% or 99.5% identity to SEQ ID NO: 4.

Primers capable of specifically amplifying fragments of 16S rRNA-encoding genes from C. nematophagum have been designed, as described in the examples below. PCR amplification using the combination CnemF1 and CnemR1 will yield a 129 bp fragment from C. nematophagum, while the combination CnemF1 and CnemR2 will yield a 394 bp fragment from C. nematophagum. Typically, at least when using the conditions described in the Materials and Methods section below, these primer pairs will not amplify any fragments from other species of Chryseobacterium, or from other bacterial genera. Where amplification is successful, the amplified sequences from Chryseobacterium nematophagum will typically have at least 98% identity to the corresponding consensus sequence from JUb129 or JUb275, e.g. at least 98.5%, 99.0% or 99.5% identity to the corresponding consensus sequence.

### Primer Sequences:

CnemF1:
(SEQ ID NO: 5)
5′ TGA TTC TTT CCC GAA TCA GA 3′
CnemR1:
(SEQ ID NO:)
5′ ATA TCA ATC GAT GCC AAT CAA T 3′
CnemR2:
(SEQ ID NO: 7)
5′ GCT TCC CAC ACG TGG AAA GG 3′

Sequences amplified by CnemF1+CnemR1 (primer sequences not included):

### JUb129 (Consensus)

(SEQ ID NO: 8)
TGATTCTTTCGGGAATCAGAGAGCGGCGTACGGGTGCGGAACACGTGTGC
AACCTGCCTTTATCTGGGGGATAGCCTTTCGAAAGGAAGATTAATACCCC
ATAATATATTGATTGGCATCGATTAATAT

### JUb275 (Consensus)

(SEQ ID NO: 9)
TGATTCTTTCGGGAATCAGAGAGCGGCGTACGGGTGCGGAACACGTGTGC
AACCTGCCTTTATCTGGGGGATAGCCTTTCGAAAGGAAGATTAATACCCC
ATAATATATTGATTGGCATCGATTAATAT

Sequences amplified by CnemF1+CnemR2 (primer sequences not included):

### JUb129 (Consensus)

(SEQ ID NO: 10)
TGATTCTTTCGGGAATCAGAGAGCGGCGTACGGGTGCGGAACACGTGTGC
AACCTGCCTTTATCTGGGGGATAGCCTTTCGAAAGGAAGATTAATACCCC
ATAATATATTGATTGGCATCGATTAATATTGAAAACTCCGGTGGATAAAG
ATGGGCACGCGCAAGATTAGATAGTTGGTGAGGTAACGGCTCACCAAGTC
CATGATCTTTAGGGGGCCTGAGAGGGTGATCCCCCACACTGGTACTGAGA
CACGGACCAGACTCCTACGGGAGGCAGCAGTGAGGAATATTGGACAATGG
GTGAAAGCCTGATCCAGCCATCCCGCGTGAAGGACGACGGCCCTATGGGT
TGTAAACTTCTTTTGTACAGGGATAAACCTTTCCACGTGTGGGAAGC

### JUb275 (Consensus)

(SEQ ID NO: 11)
TGATTCTTTCGGGAATCAGAGAGCGGCGTACGGGTGCGGAACACGTGTGC
AACCTGCCTTTATCTGGGGGATAGCCTTTCGAAAGGAAGATTAATACCCC
ATAATATATTGATTGGCATCGATTGATATTGAAAACTCCGGTGGATAAAG
ATGGGCACGCGCAAGATTAGATAGTTGGTGAGGTAACGGCTCACCAAGTC
GATGATCTTTAGGGGGCCTGAGAGGGTGATCCCCCACACTGGTACTGAGA
CACGGACCAGACTCCTACGGGAGGCAGCAGTGAGGAATATTGGACAATGG
GTGCAAGCCTGATCCAGCCATCCCGCGTGAAGGACGACGGCCCTATGGGT
TGTAAACTTCTTTTGTACAGGGATAAACCTTTCCACGTGTGGGAAGC

Table 3 (below) lists a number of genes found in Chryseobacterium nematophagum but not in other Chryseobacterium species, i.e.:

Collagenase
Chitinase
Flavastacin precursor, Astacin
Pertussis toxin subunit S1
Pertussis toxin subunit S1
Thiol-activated cytolysin (pfo)
Thiol-activated cytolysin (slo1)
Thiol-activated cytolysin (slo2)
Thiol-activated cytolysin (slo3)
Thiol-activated cytolysin (slo4)
Thiol-activated cytolysin (slo5)
Thiol-activated cytolysin (slo6)
Thiol-activated cytolysin (slo7)
Thiol-activated cytolysin (slo8)
Retroviral aspartyl protease
Protease/peptidase
D-alanyl-D-alanine carboxypeptidase precursor
D-alanine carboxypeptidase
nlpD Murein hydrolase activator
CAAX amino terminal protease
ATP-dependent Clp protease
Hemolysin (x2)

For full details and characterisation of these genes, see Ref. 44 including its additional files.

A Chryseobacterium nematophagum may contain one or more, two or more, three or more, four or more, five or more, ten or more, fifteen or more, or all of the genes listed above.

For example, the Chryseobacterium nematophagum may contain at least the collagenase, chitinase and Astacin (Flavastacin precursor) genes.

It may also contain a cysteine protease gene and one or more of the Por genes identified in Table 3. However, some or all of these are also found in at least one other Chryseobacterium species, so may be less reliable identifiers of Chryseobacterium nematophagum.

Chryseobacterium nematophagum also has the ability to kill nematodes including, but not limited to, parasitic nematodes. Without wishing to be bound by theory, it is believed that C. nematophagum is capable of killing bacterivorous nematodes after being ingested by them.

Any suitable bacterivorous nematode known to be killed by strains JUb129 or JUb275 may be used as a test species, but the free-living model species Caenorhabditis elegans (C. elegans) may be a particularly convenient model. C. nematophagum is capable of killing all life stages of C. elegans, but other species of Chryseobacterium are not, as illustrated in Table 1 below.

Compositions Containing C. nematophagum

The compositions employed in the invention contain C. nematophagum in a form suitable for delivery to the environment, plant or animal of choice. The precise format and formulation may thus vary depending on the chosen application.

The compositions may contain C. nematophagum in active form, e.g. as a crude fermentation broth, or other (e.g. more purified) form, but typically as a suspension (e.g. an aqueous suspension) of active bacteria, capable of reproducing and of killing nematodes, especially bacterivorous nematodes when ingested by them.

Alternatively, the compositions may contain C. nematophagum in lyophilised (freeze dried) form. This may be useful to facilitate storage of the compositions, regardless of the intended application. To prevent rehydration of the bacteria, such compositions may be anhydrous (e.g. oil-based) or the lyophilised bacteria may be encapsulated or otherwise coated. This may be particularly useful in compositions for administration to animals, where it may be desirable to avoid rehydration of the bacteria until they have passed through the recipient animal's digestive tract and been egested in its faeces.

Suitable materials and methods for coating or encapsulation are well known to the skilled person and include hydrophilic polymers, hydrophobic polymers, and combinations thereof, including polyvinyl alcohol, ethyl cellulose, cellulose acetate phthalate, and styrene maleic anhydride, as well as gel forming proteins (such as collagen and gelatin) and polysaccharides (such as agar, alginate such as calcium alginate, and carrageenan).

Various techniques and processes may be used for coating and encapsulation, which may be sub-divided into chemical, physiochemical, electrostatic, and mechanical processes. Chemical processes include interfacial and in situ polymerization methods. Physiochemical processes include coacervation phase separation, complex emulsion, meltable dispersion and powder bed methods. Mechanical processes include the air-suspension method, pan coating, spray drying, spray congealing, micro-orifice system and rotary fluidization bed granulator methods. Spheronization is also sometimes included with mechanical processes.

For more details, see, for example, Singh, M N et al. (2010). “Microencapsulation: A promising technique for controlled drug delivery”. Research in Pharmaceutical Sciences. 5 (2): 65-77, and references cited therein.

Preliminary experiments by the inventors indicate that C. nematophagum is capable of surviving lyophilisation and heating to 37° C. (mimicking the environment of the mammalian gut) followed by rehydration.

### EXAMPLES

Further details of the results described in this specification, including figures and additional data files, can be found in Page et al., BMC Biology (2019) 17:10 (Ref. 44), which is incorporated by reference for all purposes.

The free-living nematode Caenorhabditis elegans is an excellent genetically tractable model that has been used extensively to study nematode pathogens, the majority of which are, however, only effective against Caenorhabditis species and not against parasitic species [11]. In this study we describe the characterisation of a novel Chryseobacterium pathogen with great potential for controlling key nematode infections, including (but not limited to) nematode infections of veterinary importance. Chryseobacterium spp. are gram-negative rods, found ubiquitously in the environment with certain species being reported as having unusual matrix digesting properties [12].

In this study we searched the environment for natural nematode pathogenic bacteria in association with wild Caenorhabditid nematodes. The bacterial strain JUb129 was isolated from the free-living bacterivorous nematode Caenorhabditis briggsae from a rotten apple in Paris, France (NCBI BioSample: SAMN09925763) [13]. JUb129 was also found to display unusual pathogenic activity against C. elegans [14]. The JUb275 bacteria was subsequently isolated (December 2016) from Caenorhabditis briggsae found on a rotten fig in Bangalore, India (NCBI BioSample: SAMN09925764) [15]. Both these species were found to be highly pathogenic to C. briggsae. Additional nematode-associated Chryseobacterium and related Flavobacterium species were obtained from further environmental samples together with a plant root, amphibian and a chicken associated Chryseobacterium species. All isolates were tested for nematode killing properties against C. elegans. Of all the isolates tested, only JUb129 and JUb275 were found to kill C. elegans (Table 1).

TABLE 1
Source of Chryseobacterium and ability to kill C. elegans

| Strain | | | Nematode |
|---|---|---|---|
| ID | Strain name | Source | killing |
| JUb129 | Chryseobacterium | Orsay France, rotten apple | Yes |
| | nematophagum | | |
| JUb275 | Chryseobacterium | Bangalore, India, rotten fig | Yes |
| | nematophagum | | |
| JUb270 | Chryseobacterium | Paris, France | No |
| | shigense | | |
| JUb232 | Chryseobacterium | Plurien, France, Plums | No |
| | indoltheticum | | |
| JUb171 | Flavobacterium | Orsay, France, apple | No |
| | banpakuense | | |
| JUb166 | Flavobacterium | Orsay, France, apple | No |
| | banpakuense | | |
| JUb044 | Chryseobacterium sp. | Santeuil, France, compost | No |
| JUb043 | Flavobacterium sp. | Santeuil, France, apple | No |
| JUb022 | Flavobacterium sp. | Paris, France, flower stem | No |
| JUb007 | Chryseobacterium sp. | Le Perreux, France, | No |
| | | compost | |
| JUb001 | Flavobacterium sp. | Le Perreux, France, | No |
| | | compost | |
| 100T | Chryseobacterium | Saxony, Germany, chicken | No |
| | gallinarium | | |
| C26T | Chryseobacterium | Alabama USA, rhizosphere | No |
| | contaminans | soil | |
| — | Chryseobacterium | Glasgow, UK, Toad | No |
| | indologenes | | |

### Microbiology

Our bacteriological characterisation indicated that JUb129 and JUb275 belong to the Chryseobacterium genus. In common with other Chryseobacterium, both strains are catalase and oxidase-positive, aerobic gram-negative rods that grow on solid media to produce golden, mucoid colonies that have a pungent odour. The golden colour was shown to be due to production of a flexirubin-type pigment (Additional File 1 of Ref. 44). Both JUb129 and JUb275 were found to grow optimally at 30 C (neither grow at 37° C.) on agar plus 5% sheep blood, or tryptone soy agar plus 5% sheep blood, but also grew well but less optimally on LB agar. In liquid media, growth was more efficient in SOB media than LB media. In API 20E and API 20NE strips the strains gave positive results for gelatin, esculin and N-acetylglucosamine (Additional Files 1 and 2 of Ref. 44). The API 20E test result gave a closest identification to Chryseobacterium indologenes (86.3%).

### Phylogenetic Analysis

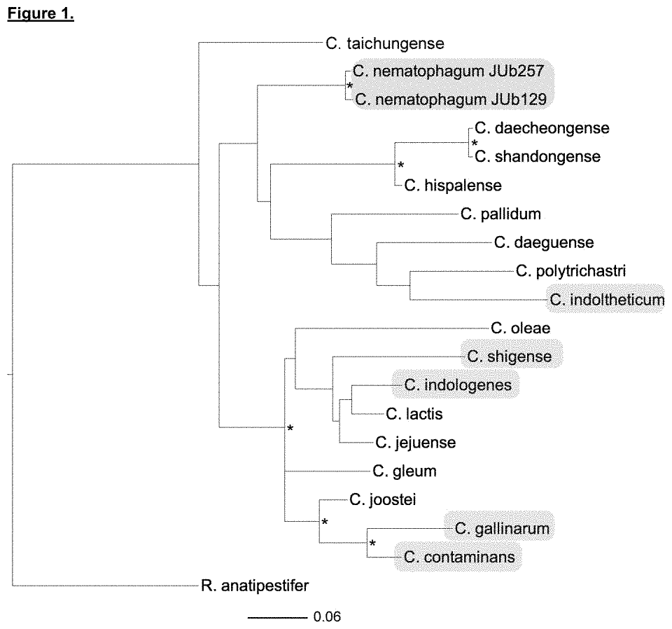

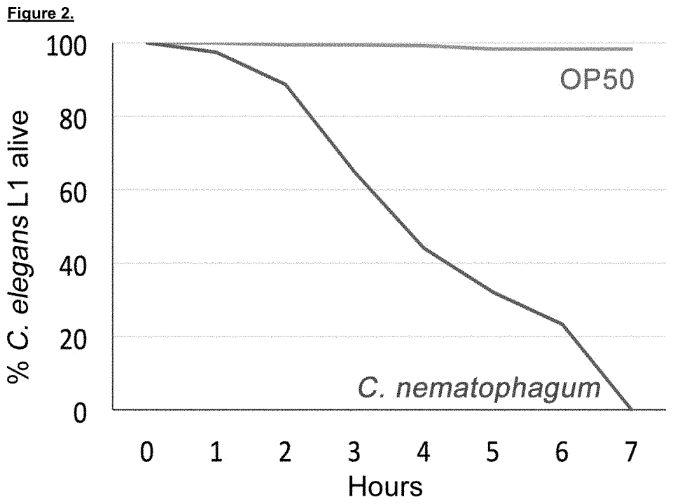

Confirmation of genus designation was obtained following whole-genome sequencing of the 4.5 Mb genomes of both the JUb129 and JUb275 isolates. The loci encoding the 16S SSU rRNA genes were identified and the sequences used to construct a phylogenetic tree (FIG. 1). The genomes of both JUb129 and JUb275 encode multiple copies of the 16S rRNA gene. JUb275 contained six identical copies of the gene while a single-nucleotide polymorphism was present in one of the six copies in JUb129. The JUb129 consensus sequence differed from the JUb275 sequence by only three nucleotides, revealing that the isolates are very similar but distinct from one other. The tree was rooted using the 16S sequence of a member of a different genus within the family Flavobacteriaceae, Reimerella anatipestifer. The JUb129 and JUb275 sequences were found to fall within the Chryseobacterium clade, indicating these isolates represents a novel species belonging to the Chryseobacterium genus, closely related to other environmental bacteria such as C. pallidum [16], C. indoltheticum [17] and C. hispalense. Consequently, we have named this new species Chryseobacterium nematophagum (from Greek crysos meaning golden and phago meaning devour; the golden nematode-devouring bacterium).

Infection and Killing of Caenorhabditis elegans

Infection experiments were carried out on staged populations of C. elegans wild type strain N2, following bleach treatment to purify and sterilise embryos. Embryos were hatched to L1 overnight in M9 buffer and synchrony of L1 larvae was initiated by feeding on either E. coli OP50-1 or JUb275. Synchronised L2, L3 and L4s were all collected from OP50-1 fed L1s. More than 50% of the L1 population were killed within three to four hours of contact with complete killing noted by seven hours exposure (FIG. 2). All larvae generally became immotile with only slight head movements following one hour of exposure to these bacilli. Similar death rates were found for L2, L3 and L4s with greater than 50% killing occurring between two and four hours (Additional File 3 of Ref. 44). Following exposure to bacteria for 48 hours, only outline traces of the larvae, representing the undigested cuticles were present on plates whereas the corresponding OP50-1 or Chryseobacterium gallinarum fed nematodes had developed further and were thriving on the bacterial food source (data not shown). Mixing experiments were set-up between the normal C. elegans food source, OP50-1, and the pathogen C. nematophagum (Additional File 4 of Ref. 44). A very low infectious dose of C. nematophagum (200 cfu) mixed with a dense population of OP50-1 (3.8×10⁷ cfu) was sufficient to kill 100% of L1s over a 24 hour exposure period (Additional File 4 of Ref. 44).

A common feature associated with many of the previously described C. elegans bacterial pathogens is the fact that the nematodes sense and are repelled from the bacterial lawns, as in the case of Pseudomonas fluorescens [18] and Serratia marcescens [19]. It significant to note that C. elegans are not repelled by C. nematophagum, but are attracted to, and remain on the bacterial lawns where they actively ingest the bacteria (Additional File 5 of Ref. 44).

A wide range of stock and environmentally-derived isolates of Chryseobacterium were tested for activity against C. elegans (Table 1), all of which lacked the unique C. elegans killing properties of JUb129 and JUb275. An example of this is C. gallinarum, isolated from a chicken, which instead provides a nutritional food source that allows full development of C. elegans.

Pharyngeal Invasion by C. nematophagum

Following exposure to the bacterial cultures on plates, C. elegans ingests C. nematophagum, which in turn multiply in the anterior pharynx and digest the nematode internally, ultimately degrading the external cuticle from the inside. Using a transgenic marker strain (VS21), encoding myo-2::mCherry that highlights the muscular pharynx, there is a progressive destruction of the anterior chitin and collagen-lined pharyngeal procorpus structure, resulting in breakdown of the anterior pharynx structure and leakage of mCherry into the anterior body cavity (data not shown).

To investigate the breakdown of the chitinous lining of the pharynx, the chitosan-specific stain eosin Y [20] was used to highlight this structure in C. elegans L1 larvae prior to exposure to C. nematophagum. Prior to bacterial exposure and one hour after exposure, eosin Y delineates the entire pharyngeal and buccal cavity linings. The pharyngeal staining and hence the chitosan structures are lost following a three hour exposure to C. nematophagum however, the buccal cavity lining remains intact (data not shown). This ability to break down the pharyngeal structure is an unusual attribute, most likely occurring through the action of specific chitinases and collagenase-like metalloproteases.

### Cuticle Collagen Degradation

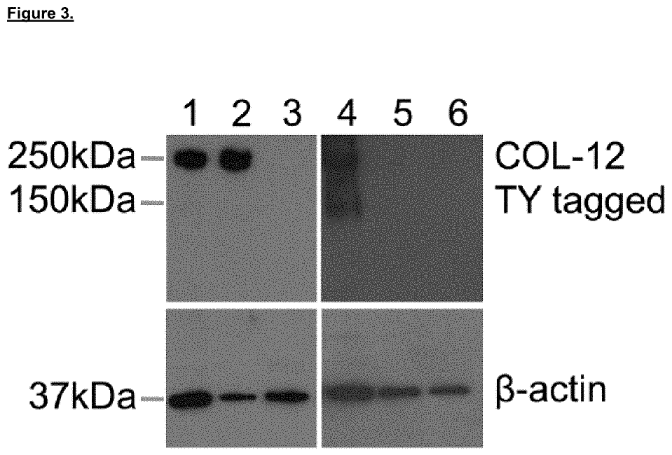

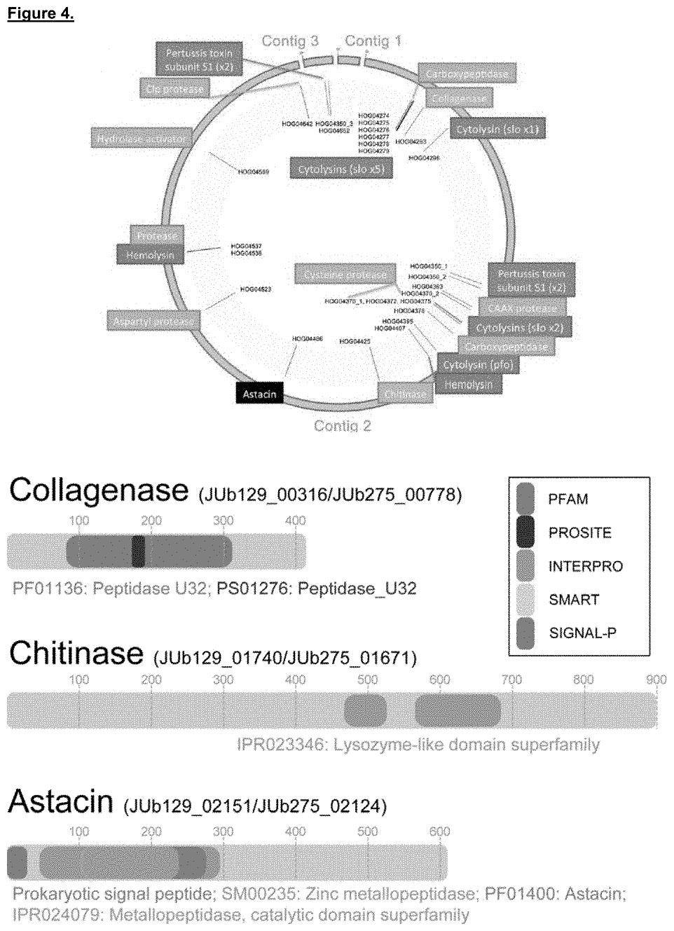

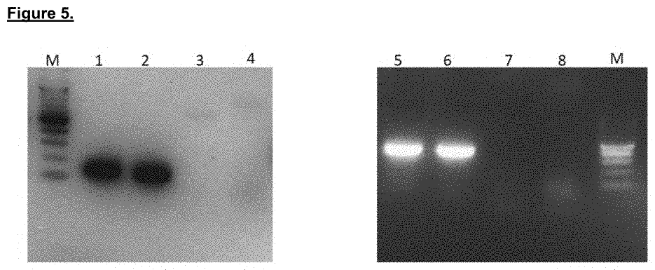

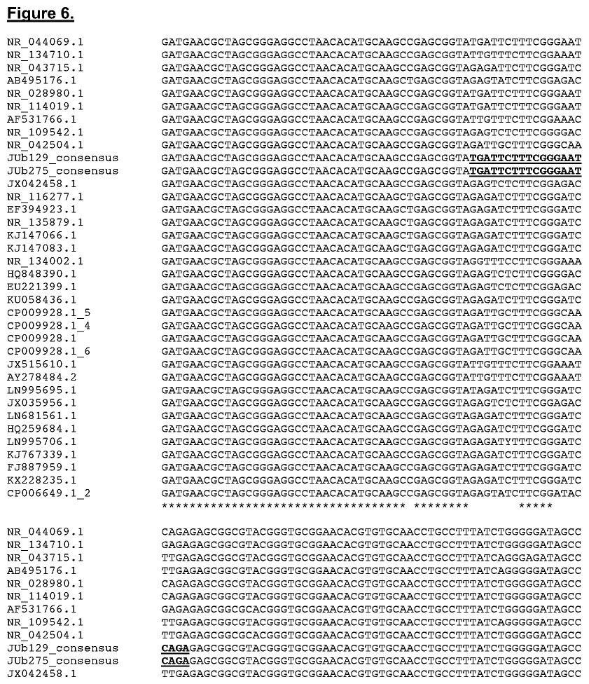

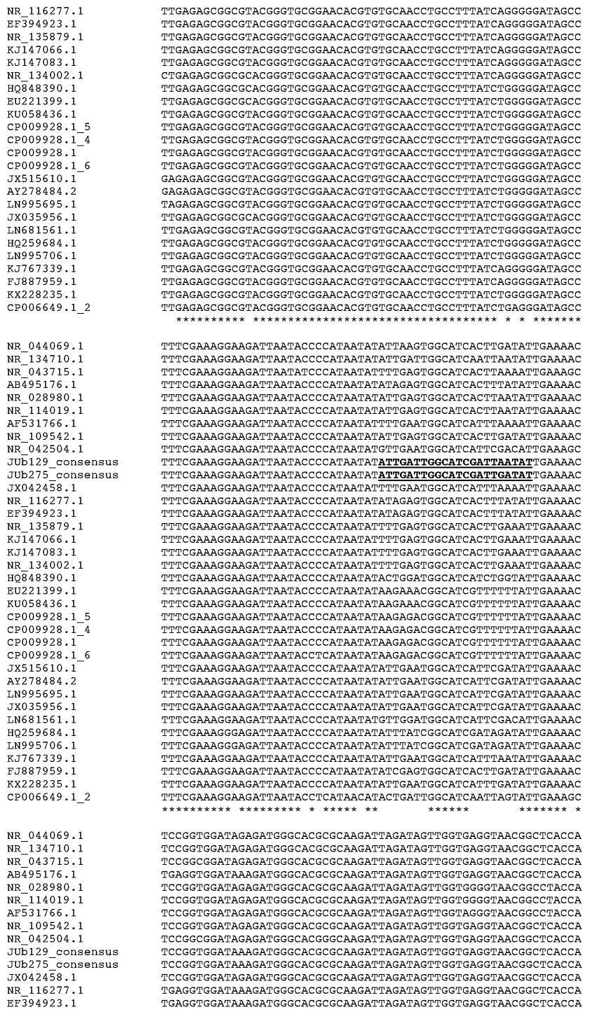

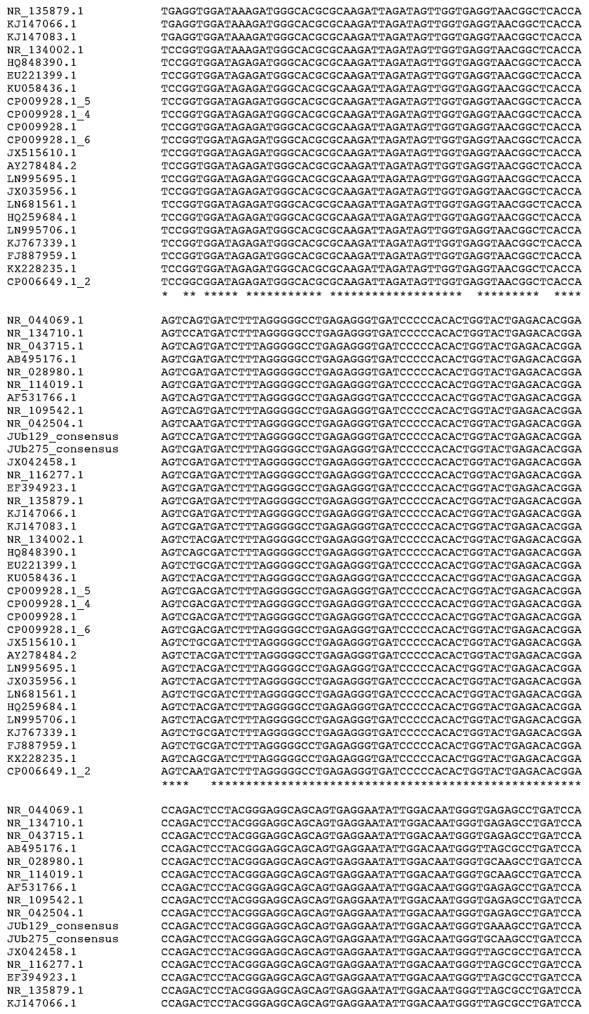

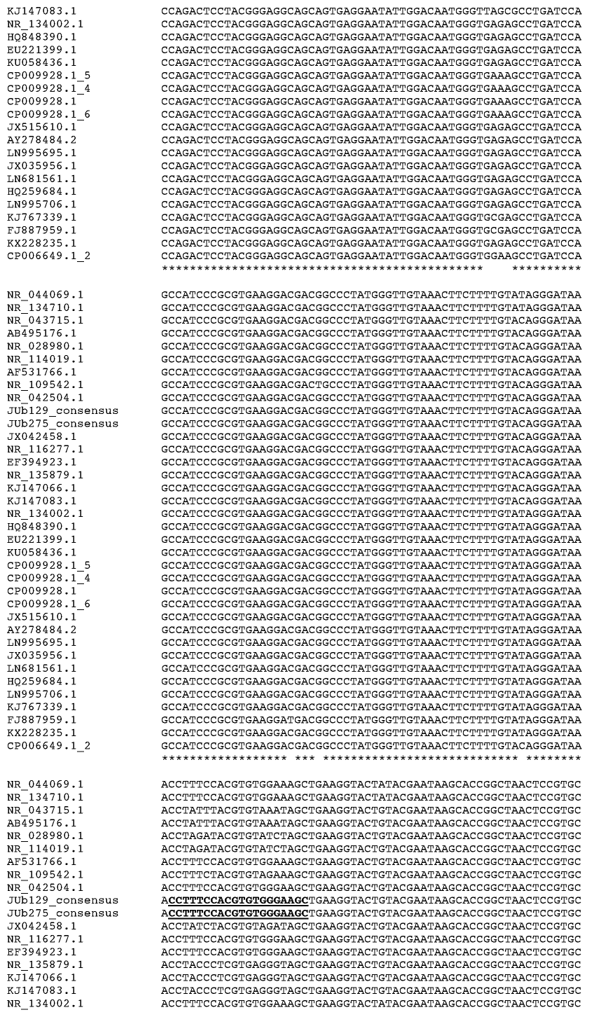

The main matrices in nematodes, most notably the cuticle and the pharynx lining, are composed of highly cross-linked collagens [21, 22]. The ability of bacteria to digest these normally insoluble structural components, especially the cuticle, is unusual and we therefore applied a TY-epitope tagged COL-12 cuticle collagen expressing strain, IA132 [23], to investigate this phenomenon. The adult C. elegans transgenic strain IA132 were incubated in the presence of C. nematophagum following culture on OP50-1 NGM plates or were cultured on pure OP50-1 plates for 24-48 hours prior to preparation of worms for Western blot analysis and probing with anti-TY tag and anti-actin antibodies. The cuticle collagen COL-12 assembles into highly insoluble non-reducible multimeric complexes in excess of 150-250 kDa (FIG. 3, lane 1). These structures are however broken-down following exposure to the C. nematophagum for 48 hours (FIG. 3, lane 3), the same samples were subsequently probed with anti β-actin, which revealed this structural protein conversely remained intact (FIG. 3, lane 3), highlighting that this digestion was specific to the COL-12 collagen. To confirm the specificity of this cuticle digestion and to exclude the possibility that OP50-1 was responsible for the cuticle collagen digestion, the following controls were carried out. 1. IA132 were incubated with non-pathogenic Chryseobacterium indologenes and 2. IA132 were also grown to adulthood on OP50-1 and were pre-cleared of OP50-1 prior to exposure to C. nematophagum. Following a 48 hour exposure of IA132 to C. indologenes, there was no degradation of multimeric tagged collagen COL-12 (FIG. 3 lane 4) and this is in contrast to the 48 hour exposure of the OP50-1 pre-cleared adults to either JUb129 or JUb275 which completely degraded the tagged collagen (data not shown).

### Parasitic Nematode Killing

As this bacterium was found to infect and kill bacterivorous nematodes, we tested it for killing activity against a number of field and laboratory isolates of parasitic nematodes including the significant Trichostrongylid and Strongylid pathogens of livestock and domesticated animals. The list of nematodes species tested is presented in Table 2 and includes animal parasites of sheep, cattle, horses, opossums, rats, wolves and a plant parasite of potatoes. All nematode stages and species were tested in a similar manner to C. elegans, namely, eggs or larvae were place on fresh bacterial lawns of C. nematophagum on NGM plates and compared to those placed on OP50-1. All free-living (L1-L3) bacterivorous stages of all nematodes tested were infected and killed by C. nematophagum in a similar manner as described from C. elegans, whereas no death was noted on OP50-1 culture. Killing rates were quantified for the L1 but not the L2 or L3 stages. Upon ingestion of bacteria the L1 larvae became immotile and 100% larvae are dead at 24 hours (Table 2). Major pathology involves infection and digestion of the pharynx followed by rupture into the body cavity and internal digestion of the nematode (Additional File 6 of Ref. 44). Infection and multiplication was investigated within the L2 larvae of Haemonchus contortus, the important trichostrongylid gastrointestinal parasite of sheep. H. contortus eggs hatch and develop from L1 to L3 stage on OP50-1 seeded NGM plates, whereas exposure of the L2 stage to C. nematophagum on NGM plates results in destruction of the anterior pharynx and eventual filling of the body cavity with bacilli (data not shown). Similar killing occurred in both anthelmintic-sensitive (ISE) and multi-anthelmintic drug resistant (IRE) strains of H. contortus. Similar infections were observed in a wide range of Strongylid and Trichostrongylid parasites (Additional File 6 of Ref. 44). The only species tested that was not killed was the potato parasitic nematode Globodera pallida (Additional File 6L of Ref. 44), and this probably reflects the non-bacterial diet and the presence of mouthparts that are specialised for piercing and feeding on plant roots. In addition, we tested this bacterial species against the larval stages of insects, namely Aedes aegypti mosquitoes and no killing or pathology was noted (Additional File 7 of Ref. 44).

TABLE 2
Nematode species killed by Chryseobacterium nematophagum

| Nematode species | | | % L1 killed in 24 hrs |
|---|---|---|---|
| (host) | Killing | Stages killed | (number counted)* |
| Caenorhabditis elegans | + | All stages | 100% (0/193) |
| (free-living) | | | |
| Caenorhabditis briggsae | + | All stages | Not determined |
| (free-living) | | | |
| Globodera pallida | − | None | Not determined |
| (potato) J2 and J3 | | | |
| Haemonchus contortus | + | L1, L2 and L3 | 100% (IRE 0/215) |
| ISE and IRE strains | | | |
| (sheep and goats) | | | |
| Trichostrongylus vitrinus | + | L1, L2 and L3 | 100% (0/56) |
| (sheep and goats) | | | |
| Teladorsagia | + | L1, L2 and L3 | 100% (0/201) |
| circumcincta | | | |
| (sheep and goats) | | | |
| Cyathastomin sp. | + | L1, L2 and L3 | 100% (0/143) |
| (horses) | | | |
| Ostertagia ostertagi | + | L1, L2 and L3 | 100% (0/158) |
| (cattle) | | | |
| Parastrongyloides | + | All free-living | Not determined |
| trichosura | | stages | |
| (opossum) | | | |
| Cooperia curtecei | + | L1, L2 and L3 | 100% (0/47) |
| (sheep and goats) | | | |
| Cooperia oncophera | + | L1, L2 and L3 | 100% (0/54) |
| (cattle) | | | |
| Nippostrongylus | + | L1, L2 and L3 | 100% (0/314) |
| brasiliensis | | | |
| (rats and mice) | | | |
| Ancylostoma caninum | + | L1, L2 and L3 | 100% (0/44) |
| (dogs, wolves and foxes) | | | |

* 24 hour survival rate relates to number of freshly hatched L1s larvae added to NGM plates seeded with Chryseobacterium nematophagum that have survived after 24 hour culture period. (number surviving 24 hours/number of L1 added).

### Comparative Genomics

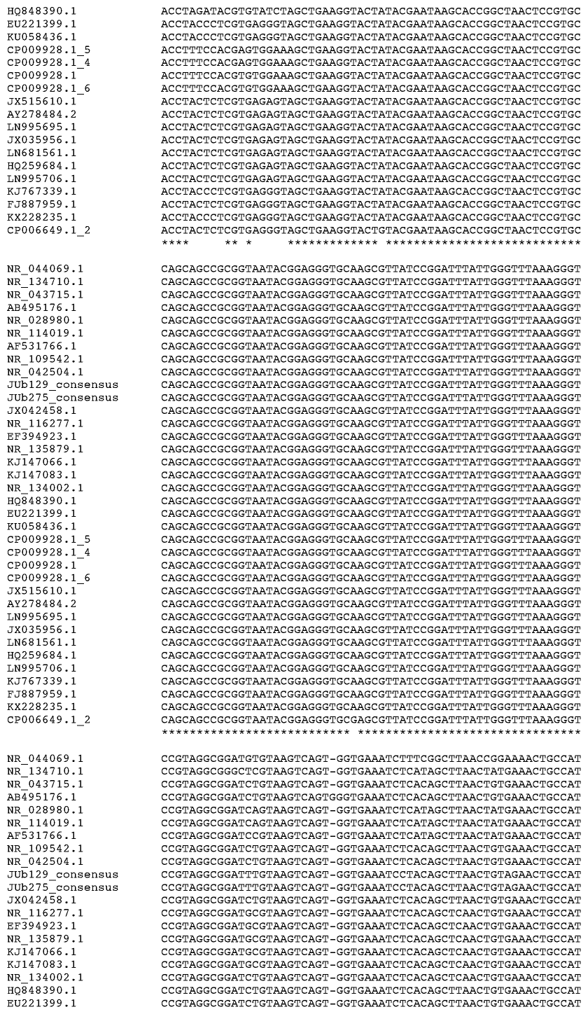

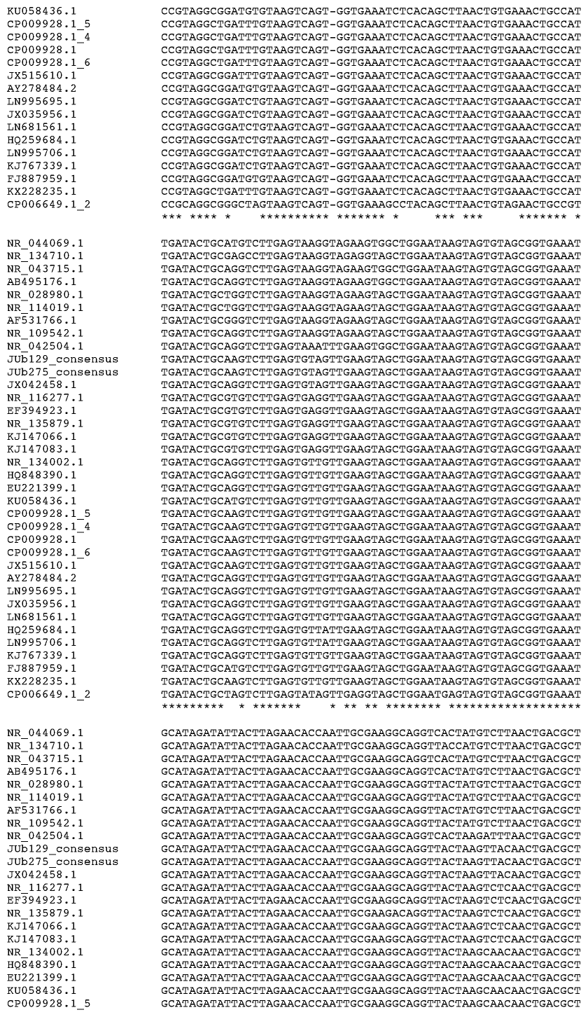

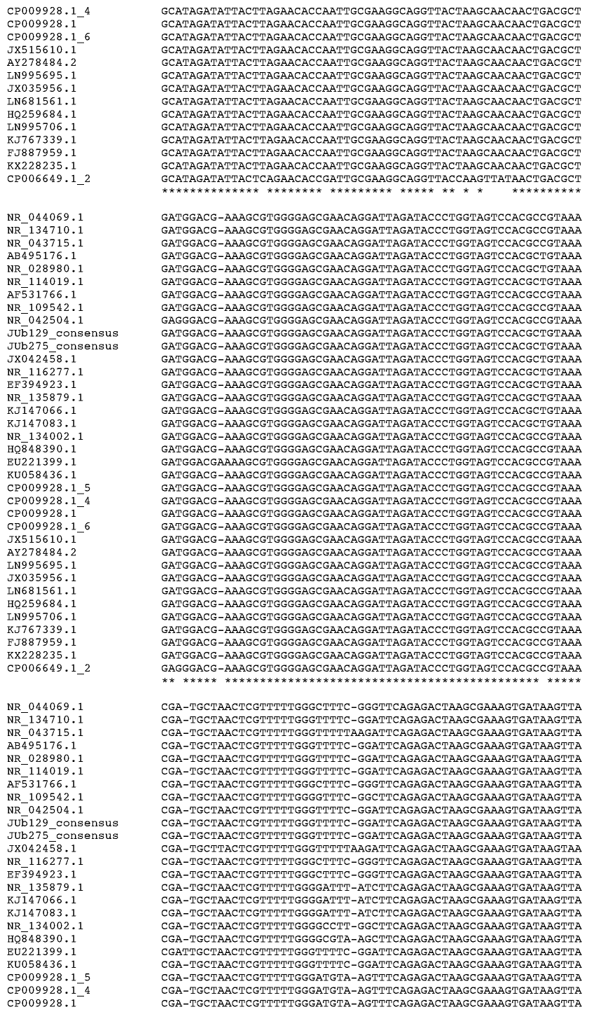

The genomes of JUb129 and JUb275 were predicted to encode 3,738 and 3,586 protein sequences, respectively. Annotated genomic sequence files are available in Additional Files 8 and 9 of Ref. 44. In order to investigate which genes might be involved in conferring the nematode-killing ability of C. nematophagum, the two genomic sequences representing this species were compared to that of five other Chryseobacterium spp. known not to possess the nematode-killing phenotype. A total of 5,020 sets of orthologous genes were identified, which were organised into 4,657 hierarchical orthologous groups (HOGs), detailed in Additional File 10 of Ref. 44. Only seventy-seven HOGs represented in JUb275 were not detected in JUb129 (1.66%), while only 136 HOGs represented in JUb129 were not detected in JUb275 (2.92%), illustrating the high degree of similarity between these two annotated assemblies. The entire set of HOGs was screened to identify which ones were specific to or expanded within C. nematophagum. 382 such HOGs were identified (Additional File 11 of Ref. 44), representing about 13% of the C. nematophagum genome, the majority of which were identified as C. nematophagum-specific. The ability to digest the nematode cuticle and the pharyngeal lining are unusual properties for a bacterium and predicted to be carried out by specific collagenases and chitinases. In order to identify these together with other genes of interest, a further subset of HOGs was identified where gene annotation contained terms such as ‘protease’, ‘peptidase’, ‘collagenase’, ‘chitinase’, ‘gelatinase’, ‘lysin’ or ‘toxin’. This resulted in the identification of 24 high-value candidate HOGs (Table 3). The genomic locations of these gene are illustrated in FIG. 4A and Additional File 12 of Ref. 44. These candidate genes include C. nematophagum-specific collagenase, chitinase and astacin encoding genes; the primary domain structure of these three key enzymes is illustrated in FIG. 4B. The collagenase enzyme is a 414 amino acid protein that is completely conserved at the amino acid level between the two C. nematophagum isolates. The chitinase is an 899 amino acid protein that has 93% identity while astacin comprises 610/614 amino acid residues, sharing 92% identity between isolates. The astacin protein contains an N-terminal prokaryotic secretion signal indicated it is secreted across the inner membrane.

TABLE 3
Nematode-killing candidate genes

| | | Number | | |
|---|---|---|---|---|
| Hierarchical | | of copies | | |
| Orthologous | | JUb | JUb | PFAM |
| Group (HOG) | Annotation | 129 | 275 | domain |
| HOG04283 | Collagenase | 1 | 1 | PF01136 |
| HOG04425 | Chitinase | 1 | 1 | — |
| HOG04486 | Flavastacin precursor, Astacin | 1 | 1 | PF01400 |
| HOG04350 | Pertussis toxin subunit S1 | 3 | 1 | — |
| HOG04652 | Pertussis toxin subunit S1 | 1 | 1 | — |
| HOG04395 | Thiol-activated cytolysin (pfo) | 1 | 1 | PF01289 |
| HOG04296 | Thiol-activated cytolysin (slo1) | 1 | 1 | PF01289 |
| HOG04279 | Thiol-activated cytolysin (slo2) | 1 | 1 | PF01289 |
| HOG04278 | Thiol-activated cytolysin (slo3) | 1 | 1 | PF01289 |
| HOG04277 | Thiol-activated cytolysin (slo4) | 1 | 1 | PF01289 |
| HOG04276 | Thiol-activated cytolysin (slo5) | 1 | 1 | PF01289 |
| HOG04275 | Thiol-activated cytolysin (slo6) | 1 | 1 | PF01289 |
| HOG04372 | Thiol-activated cytolysin (slo7) | 1 | 1 | PF01289 |
| HOG04375 | Thiol-activated cytolysin (slo8) | 1 | 1 | PF01289 |
| HOG04370 | Cysteine protease, C1A family^{a} | 2 | 2 | PF00112 |
| HOG04523 | Retroviral aspartyl protease | 1 | 1 | PF00077 |
| HOG04537 | Protease/peptidase | 1 | 1 | PF00664 |
| HOG04274 | D-alanyl-D-alanine | 1 | 1 | PF00144 |
| | carboxypeptidase precursor | | | |
| HOG04378 | D-alanine carboxypeptidase | 1 | 1 | PF00144 |
| HOG04589 | nlpD Murein hydrolase activator | 1 | 1 | PF01551 |
| HOG04363 | CAAX amino terminal protease | 1 | 1 | PF02517 |
| HOG04642 | ATP-dependent Clp protease | 1 | 1 | PF00004 |
| HOG04407 | Hemolysin | 1 | 1 | PF12700 |
| HOG04538 | Hemolysin | 1 | 1 | — |
| 24 x HOGs | Por gene^{b} | 38 | 38 | PF00041; |
| | | | | PF07593; |
| | | | | PF11958; |
| | | | | PF04231; |
| | | | | PF01421; |
| | | | | PF02128 |

^{a}single copy present in C. shigense genome;
^{b}also present in other Chryseobacterium spp. genomes: C. contaminans (n = 8), C. gallinarum (n = 7), C. indologenes (n = 6), C. indoltheticum (n = 4) and C. shigense (n = 6).

Interestingly, the C. nematophagum genome contains two Pertussis toxin S1 subunit-encoding genes, one of which has expanded to three paralogues in JUb129. The presence of these toxin-encoding genes was assessed across all 120 Chryseobacterium spp. genomes currently available in the NCBI database and these were found to be absent, indicating they are likely the result of a lateral gene transfer event in the recent evolutionary history of this species. Strikingly, and also specific to the C. nematophagum genome, nine members of a thiol-activated cytolysin family, the s/o genes, were identified.

Analysis of the genome of C. nematophagum has allowed us to identify all the major components of the gliding and PorS/Type IX secretion system genes; with orthologues of GldA, B, C, D, E, F, G, H, J, K, L, M, N and SprT and SprA all being present. Interestingly, the PorS genes GldK, GldL, GldM and GldN are found clustered in a single region in the genome (Additional file 12 of Ref. 44), perhaps representing an operon. This secretion system is known to be associated with virulence and gliding motility in the phylum Bacteroidetes, of which Chryseobacterium is a member.

Chryseobacterium nematophagum Diagnostic PCR.

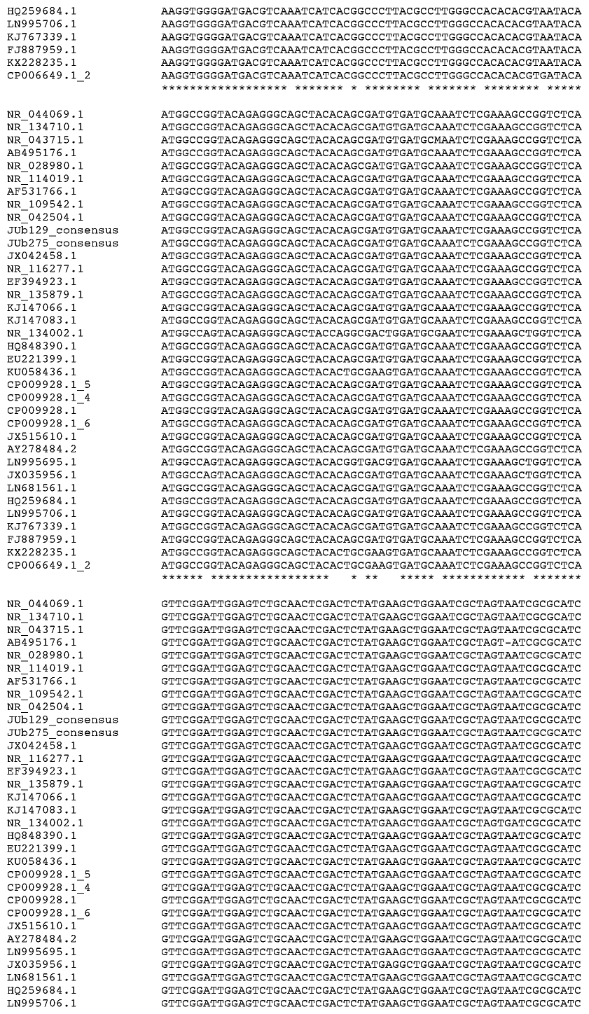

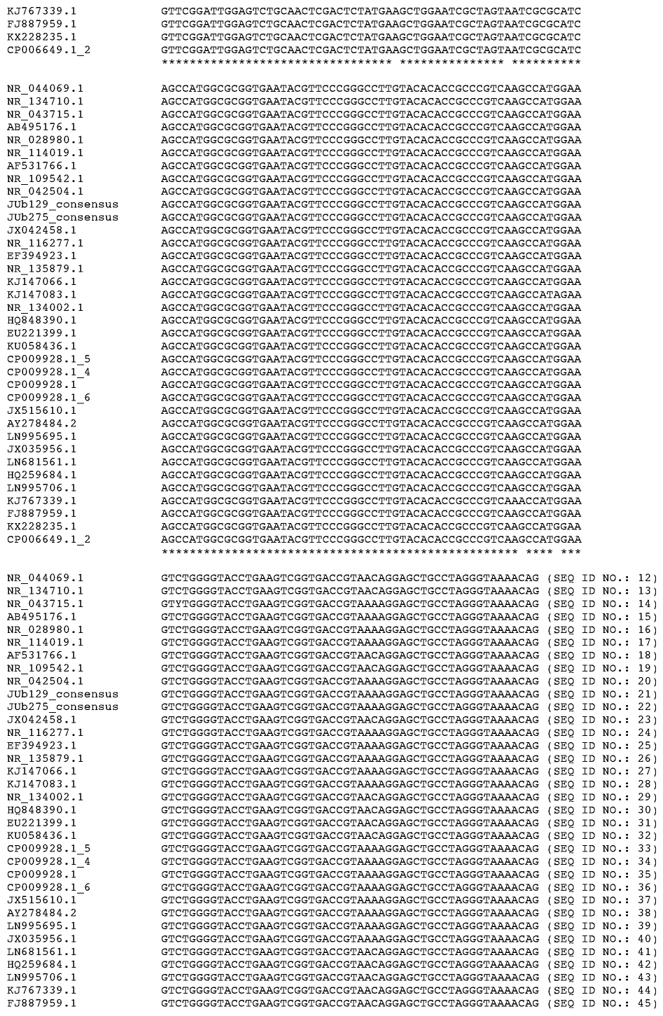

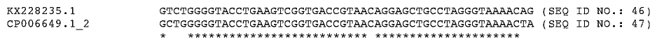

16S ribosomal RNA encoding genes from C. nematophagum European isolate (JUb129) and Asian isolate (JUb275) were aligned with 16S rRNA sequences from 35 other available environmental Chryseobacterium species (FIG. 6). Primers specific to JUb275 and JUb129 were designed:

CnemF1
5′ TGA TTC TTT CCC GAA TCA GA 3′
CnemR1
5′ ATA TCA ATC GAT GCC AAT CAA T 3′
CnemR2
5′ GCT TCC CAC ACG TGG AAA GG 3′

The combination of CnemF1 and CnemR1 amplifies a 129 bp fragment that is 100% identical between JUb275 and JUb129. The combination of CnemF1 and CnemR2 amplifies a 394 bp fragment that is 99.75% identical between JUb275 and JUb129.

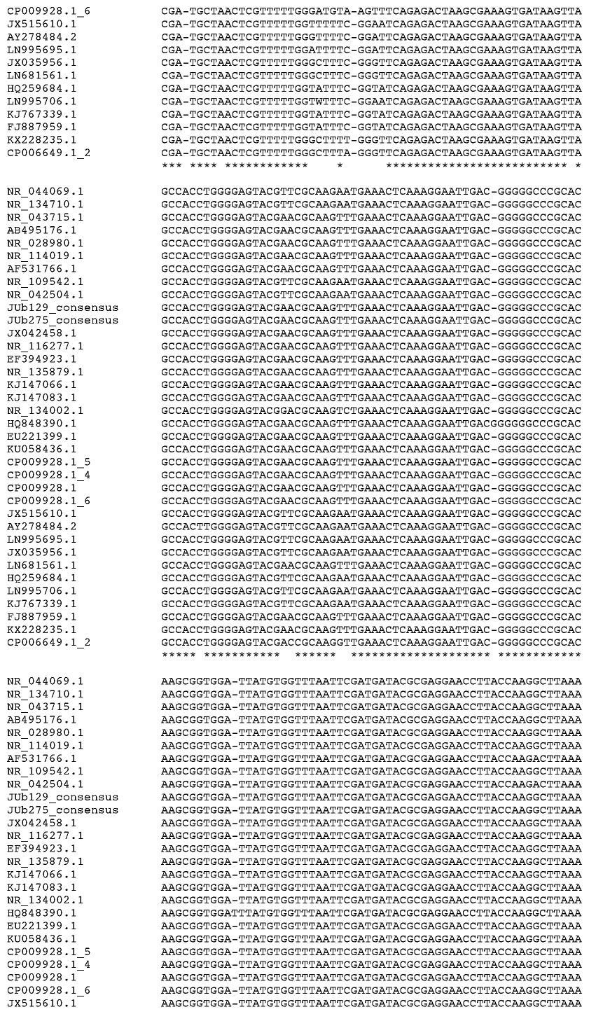

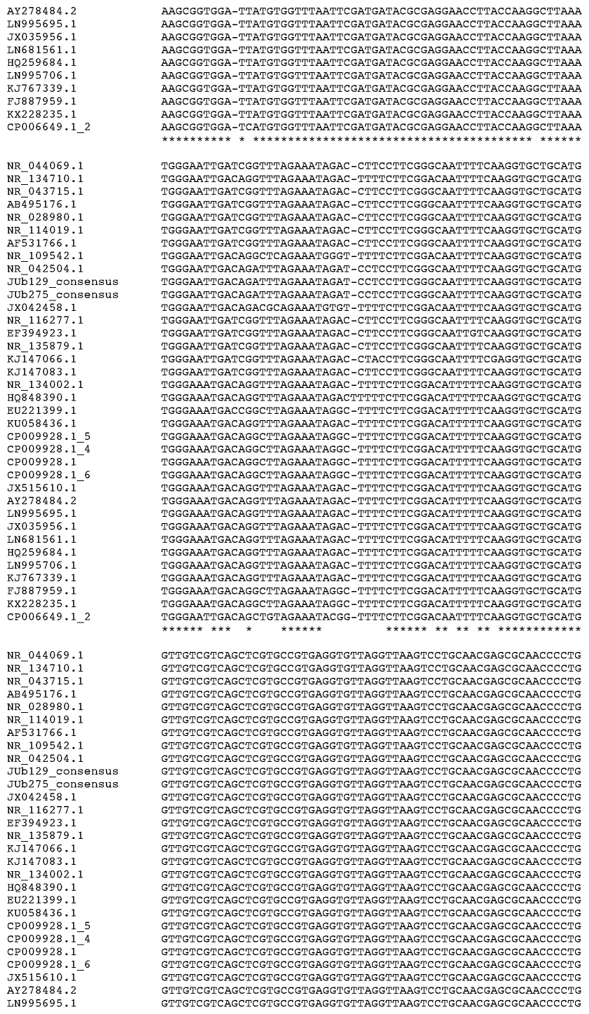

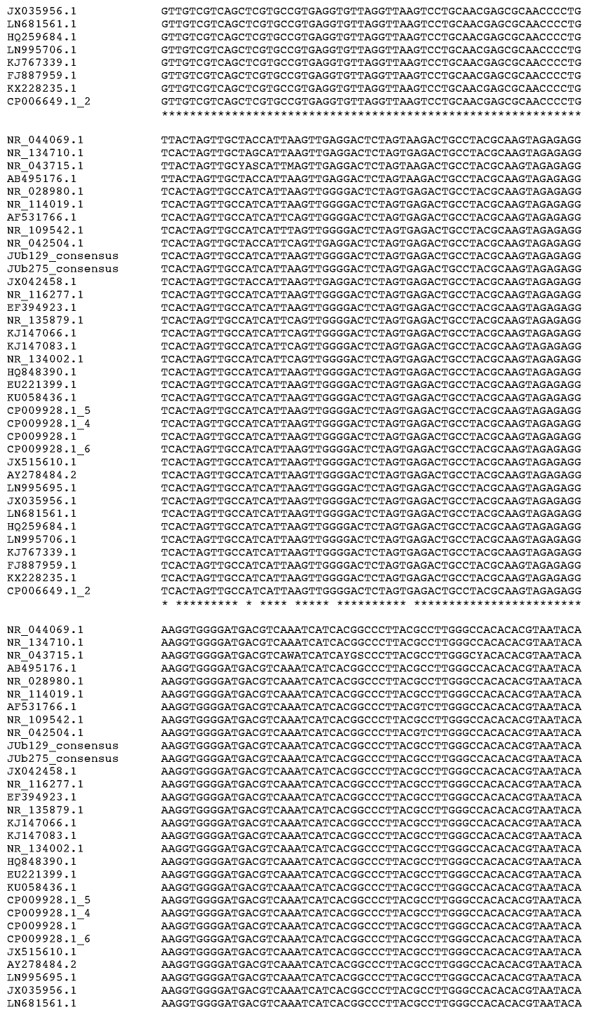

These primer combinations demonstrate clear amplification of appropriate sized bands from C. nematophagum isolates but not from either Chryseobacterium indologenes or Escherichia coli (FIG. 5).

### Discussion

In this study we have identified and characterised a novel environmental Chryseobacterium species with potent nematocidal properties that we have named Chryseobacterium nematophagum. Two separate, but very closely related isolates, were isolated from Europe (Paris) and Asia (Bangalore), both were found associated with and colonising the free-living nematode Caenorhabditis briggsae, and both isolates of infected nematodes were found associated with rotting fruit and the accompanying bacterial flora. Chryseobacterium nematophagum rapidly kills both C. briggsae and the sister species C. elegans, but more significantly this Chryseobacterium infects and kills all bacterivorous stages of all the parasitic nematodes tested to date, indicating that if ingested these bacteria will kill and colonise many nematode species. Nematode killing was rapid, occurring within three hours of ingestion and involved the digestion of the anterior pharyngeal lining followed by bacteria invasion and colonisation of the body cavity. All nematode tissues were ultimately consumed, including the normally highly insoluble cuticular exoskeleton. Both the pharynx and the cuticle are composed of large highly crosslink non-reducible collagens [21, 22] and complex carbohydrate macromolecules such as chitin [24].

Digestion of these matrix materials requires the action of active collagenase and chitinase enzymes, members of which are uniquely encoded in the genome of the pathogenic bacteria analysed in this study. Nevertheless, it is interesting to note that chitinase, gelatinase and collagenase metalloprotease activities have all been described in related Chryseobacterium species and have been linked with gliding motility, PorS type IX secretory systems and virulence characteristics [25]. It is also significant to note that these bacteria have neither collagen nor chitin proteins or structures. Chryseobacterium species belong to the Bacteriodetes phylum, members of which are being increasingly describes as having unusually linked motility (gliding, Gld) and secretory system (PorS, Spr) [25, 26]. It is also significant to note that one of the two chitinases also possess a C-terminal PorS domain, indicating that this enzyme should be secreted from this bacterium. This PorS type IX secretory system differs from the well-defined type I-VI bacterial secretory systems and differs from the mycobacterial type VII system and the type VIII systems [26]. The C. nematophagum gliding and PorS components are very similar to those of other Bacteroidetes such as Flavobacterium johnsoniae [25]. The importance of these PorS-secreted digesting enzymes is clearly demonstrated in Chryseobacterium sp. strain kr6, which was isolated from poultry industry waste and found to degrade chicken feathers via the action of a specific keratinase enzyme [12].

Analysis of the C. nematophagum genome has identified genes that encode additional PorS-secreted proteins that may be involved in matrix digestion and virulence, with the secreted astacin metalloproteases, chitinases and collagenases being particularly good candidates for future characterisation with respect to virulence.

By applying a C. elegans transgenic collagen reporter strain we have also demonstrated by Western Blotting that this bacterium can digest the highly insoluble cross-linked cuticle collagens (FIG. 3). Additionally, by analysing an mCherry pharyngeal transgenic marker strain and a chitosan-specific stain we can observe the physical destruction of this collagen and chitin-lined structure which occurs in less than three hours.

### CONCLUSIONS

We have investigated the ability of C. nematophagum to kill and digest the environmental stages of field isolates of important Trichostrongyle and Strongyle nematodes of livestock and domesticated animals. Invasion and digestion proceeds in a similar fashion to that described for the model nematode C. elegans. Most notably, C. nematophagum kills the environmental L1-L3 stages of an anthelmintic resistant strain (IRE) of the sheep parasite Haemonchus contortus. This bacterium is also highly effective against the L1-L3 stages of the horse Cyathostomins, a group of Strongyle nematodes that are becoming increasingly resistant to all the available anthelmintic classes. This pathogenicity raises the possibility that C. nematophagum, or indeed its isolated virulence factors, could provide a future novel means of controlling these increasingly problematic parasites of grazing livestock. Ultimately, it may also provide an alternative control measure to fight the pathogenic soil transmitted helminths of humans, including, for example, the important hookworm parasites, Ancylostoma duodenale and Necator americanus, both of which are related to the wolf hookworm Ancylostoma caninum and rat hookworm N. brasilliensis, which are both highly susceptible to killing by this bacterium. We find that C. nematophagum grows efficiently in the presence of nematodes and that C. elegans are attracted to and not repelled by this bacterium, suggesting that this represents a true host-pathogen interaction.

### Methods

### Nematode Strains, Culture and Killing Assays

C. elegans N2, VS21 [myo-2p::mCherry] and C. briggsae (AF16) strains were supplied by the C. elegans Genetics Centre (CGC). The TY-tagged col-12 strain IA132 was provided by lain Johnstone (University of Glasgow). All Caenorhabditis strains were maintained on NGM agar plates supplemented with E. coli bacteria OP50-1 following standard techniques http://www.wormbook.org/toc_wormmethods.html.

The lab-derived drug sensitive Haemonchus contortus strain MHco3(ISE) and drug resistant MHco18(IRE), and field isolates of the following Trichostrongylids H. contortus, Trichostrongylus vitrinus, Teladorsagia circumcinta, Ostertagia ostertagi, Cooperia curticei and Cooperia oncophera were all kindly provided by Dave Bartley and Alison Morrison from the Moredun Research Institute. Additional field isolates of T. circumcincta were provided by George King (University of Glasgow) and the Cyathastomin parasites were provided by Ronnie Barron (University of Glasgow). Globodera pallida J2 larvae were provided by Aaron Maule (Queens University), Nippostrongylus brasilliensis was proved by Rick Maizels (University of Glasgow), Ancylostoma caninum was provided by Elizabeth Schmidt (Botucatu, Brazil) and Parastrongyloides trichosura was provided by Adrian Streit (Max-Planck, Tuebingen). For the Trichostrongylids and Strongylids, embryos were purified from faecal samples derived from either mono-specifically infected donor lambs or naturally infected ruminants, via saturated salt flotation. The eggs were hatched to L1 and developed to L2 and L3 by culturing on NGM agar OP50-1 as per C. elegans. Free-living P. trichosura and Globodera pallida J2 larvae were cultured on NGM plates as per C. elegans.

### Nematode Killing Assays

All nematode stages and species were tested by placing either 100 freshly prepared eggs or 50-100 L1 larvae on fresh bacterial lawns (200 μl of overnight culture in SOB media) of C. nematophagum on 5 cm NGM plates and survival and morphology was observed over two to three days and compared to those placed on OP50-1. Time-course experiments were carried out on L1 larvae derived from bleach treated hermaphrodite egg preparation (10 mins 250 mM KOH/1% bleach). Eggs were washed and allowed to hatch overnight in the absence of bacteria, then L1s were added to bacterial lawns on NGM plates. To obtain synchronised L2, L3 and L4s, the larvae were collected at various time-points from OP50-1 fed L1s. Likewise, the pharyngeal labelled strain, VS21, was bleach treated to derive eggs then L1s that were observed for pharyngeal damage via U.V. microscopic analysis.

### Chitosan Staining

The chitosan staining protocol followed a modified version of a previously published method [20]. Briefly, freshly prepared L1s of C. elegans were washed then suspended in 500 μl citrate phosphate buffer, pH6 (0.2M NaH₂PO₄ and 0.1M K citrate) prior to adding 15 μl eosin Y stock (5 mg/ml in 70% ethanol). Tubes were incubated in the dark for 10 mins, then washed extensively in citrate phosphate buffer before adding to C. nematophagum inoculated NGM plates. Samples were observed and images collected over a six hour period.

### Imaging and Microscopy

All nematodes were either viewed on plates using a Zeiss bench-top microscope fitted with a Canon Sureshot camera or were mounted on 2% agar pads on slides and viewed under Differential Interference Contrast (DIC) or fluorescence optics on a Zeiss Axioscop2 and imaged with a Zeiss AxioCam camera and Axiovision software.

### DNA Extraction and Whole Genome Sequencing

Overnight cultures of JUb129 and JUb275 each in 10 ml SOB were re-suspended in 2 ml 25% sucrose in TE, digested in 100 mg/ml Roche lysozyme followed by 20 mg/ml proteinase K digestion. 10 mg/ml RNAse was added prior to adding 400 μl 0.5M EDTA and 30 μl of 10% sarcosyl and leaving at 50° C. overnight. Samples were made up to 12 mls with TE prior to phenol/chloroform extraction and ethanol precipitation. Samples were then sent to Wellcome Trust Sanger Institute for whole genome sequencing using the PacBio platform.

For JUb129, PacBio RS sequence reads were assembled using HGAP v3 [27] of the SMRT analysis software package v2.3.0. The fold coverage was set to 30 and the approximate genome size was set to 3 Mb. The assembly was circularised using Circlator v1.1.3 [28] and polished using the PacBio RS resequencing protocol and Quiver v1 [27] of the SMRT analysis software package v2.3.0. For JUb275, PacBio RSII reads were converted to BAM format using the SMRTlink pipeline v5.0.1.9585 and then to FASTQ format using Samtools v1.6 [29], excluding reads which failed quality control. Reads were assembled using CANU v1.6 [30]. The assembly was circularised using Circlator v1.5.3 [28]. The PacBio SMRTlink resequencing pipeline was run utilising Quiver [27] and the corrected reads mapped back to the final assembly using minimap2 v2.6 [31]. For both genomes, automated annotation was undertaken using PROKKA v1.11 [32] based on a genus-specific NCBI Reference Sequence (RefSeq) database. Protein motifs were identified on the basis of matching domains in the UniProtKB [33], TIGRFAM [34], PFAM [35] and NCBI protein cluster [36] databases. Genomic sequence data has been deposited in the NCBI database under BioProject number PRJNA487926 and BioSamples numbered SAMN09925763 (JUb129) and SAMN09925764 (JUb275). Annotated genomic sequence data is available in Additional File 8 (JUb129) and Additional File 9 (JUb275) of Ref. 44.

### Phylogenetics

A maximum likelihood tree, based on the 16S SSU rRNA gene, was generated using RAXML [37] using a generalised time-reversible model of sequence evolution. The tree was constructed using the JUb129 and JUb275 sequences together with a representative collection of Chryseobacterium spp. sequences downloaded from the NCBI database, including that of C. lactis (LN995695.1), C. indologenes (JX515610.1), C. jejuense (JX035956.1), C. shigense (NR 041252.1), C. oleae (NR 134002.1), C. contaminans (NR 133725.1), C. gallinarum (CP009928.1), C. gleum (FJ887959.1), C. joostei (KU058436.1), C. shandongense (NR 135879.1), C. daecheongense (KJ147083.1), C. hispalense (NR 116277.1), C. daeguense (NR 044069.1), C. polytrichastri (NR 134710.1), C. indoltheticum (NR_042926.1), C. pallidum (NR_042504.1) and C. taichungense (JX042458.1). The 16S sequence of another member of the Flavobacteriaceae family, Riemerella anatipestifer (CP006649.1_2), was used as an out-species to root the tree. Stability was assessed using 100 bootstrap pseudo-replicates and the tree was visualised using FigTree 1.4 (http://tree.bio.ed.ac.uk).

### Comparative Genomics

Orthologous genes were defined across the genomes of JUb129, JUb275 along with five Chryseobacterium spp. known to not possess the nematode-killing phenotype, namely C. contaminans (GCA_001684955.1), C. gallinarum (GCA_001021975.1), C. indologenes (GCF_001295265.1), C. indoltheticum (GCA_900156145.1) and C. shigense (GCA_900156575.1). Annotated genomic sequence data for these species was downloaded from the NCBI Genome database. Pairs of orthologous sequences were identified using a stand-alone version of the Orthologous Matrix (OMA) algorithm v2 [38], following which hierarchical orthologous groups were inferred [39] (Additional File 11 of Ref. 44). Top-ranking ‘nematode-killing’ candidate genes were analysed for known amino acid motifs using InterProScan [40]. These sequences were then compared to the 120 Chryseobacterium spp. genomes currently available in NCBI Genome database (Additional File 13 of Ref. 44) using the BLASTP 2.2.26+ [41].

Analysis of Collagenase Activity of C. nematophagum

Twenty young adults of the COL-12 TY tagged C. elegans strain (IA132) were picked from OP50-1 NGM plates and incubated in microtitre wells containing 100 μl M9 buffer and 10 μl C. nematophagum broth for 24-48 hrs with controls being identically treated IA132 with OP50-1 or Chryseobacterium indologenes for 48 hours. To exclude the involvement of OP50-1 in collagen digestion, a set of experiments included adult IA132 worms grown on OP50-1 which were washed three times in M9 and cultured for four hours on unseeded NGM plates prior to culturing with JUb129 and JUb275 for 48 hours. All wells were set-up in triplicate and well contents were transferred to Eppendorfs and centrifuged 1000 rcf for two minutes and the pellets frozen at −20° C. Pelleted worms were resuspended in 1×SDS PAGE sample buffer with 5% mercaptoethanol and boiled for ten mins, centrifuged and supernatant added to wells of 4-20% mini-protean Bio-Rad SDS PAGE gels and run at 200v for 30 minutes. Western blotting was carried out on a Bio-Rad Mini Transfer Cell following the manufacturer's recommendations. The PVDF membrane (GE Healthcare) was removed, blocked in 5% marvel PBS 0.1% tween, probed with anti-TY tag and then Goat anti-mouse HRP and this was followed by detection with Pierce ECL plus substrate. Blots were then stripped and re-probed with anti-actin antibody.

### Microbiology

Analytical Profile Index (API) strips (20E and 29NE) were analysed following the manufacturer's instruction (Biomerieux) by incubating at 30° C. for 24 and 48 hrs. Oxidase tests were carried out by selecting a colony of C. nematophagum on a cotton bud and adding drops of tetramethyl-p-phenylenediamine dihyrochloride. Gram staining was performed on a slide spread of C. nematophagum following conventional methods [42]. Flexirubin tests were performed on C. nematophagum colonies using 20% KOH as described [43].

Chryseobacterium nematophagum Diagnostic PCR.
16S rRNA Primer Sequences:

CnemF1:
5′ TGA TTC TTT CCC GAA TCA GA 3′
CnemR1:
5′ ATA TCA ATC GAT GCC AAT CAA T 3′
CnemR2:
5′ GCT TCC CAC ACG TGG AAA GG 3′

Single colonies of freshly grow bacteria were picked into 100 μl of distilled water, boiled for 10 minutes and centrifuged for 10 minutes. 5 μl of this supernatant was added to the following PCR reactions:

### For 129 bp Fragment:

10 pMol CnemF1
10 pMol CnemR1
12.5 mM each of dATP, dCTP, dTTP, dGTP.

### 1 μl 25 mM MgCl₂

5 μl 5× GoTaq reaction buffer (Promega M7808)
Distilled water to 10.5 μl
2.5 U GoTaq G2 Flexi DNA polymerase (Promega M7808)

### For 394 bp Fragment:

10 pMol CnemF1
10 pMol CnemR2
12.5 mM each of dATP, dCTP, dTTP, dGTP.

### 1 μl 25 mM MgCl₂

5 μl 5×GoTaq reaction buffer (Promega M7808)
Distilled water to 10.5 μl
2.5 U GoTaq G2 Flexi DNA polymerase (Promega M7808)

Reactions were cycled for 30 rounds on PCR thermocycler using the following conditions: 92° C. for 1 minute; 53° C. for 1 minute; 72° C. for 1 minute.

### Deposits Under the Budapest Treaty

Deposits of C. nematophagum strains JUb129 and JUb 275 have been deposited under the terms of the Budapest Treaty as follows:

### Strain:

Chryseobacterium nematophagum strain JUb129

### Depository Institution:

### CABI (Centre for Agriculture and Bioscience International)

### CABI Bioscience, UK Centre (IMI)

### Bakeham Lane

### Englefield Green

### Egham

### Surrey

### TW20 9TY

Date of Deposit: 4 Jun. 2019; accepted 11 Jun. 2019

### Accession Number: IMI CC Number 507105

Depositor: Prof. Antony Page, on behalf of The University Court of the University of Glasgow

### Strain:

Chryseobacterium nematophagum strain JUb275

### Depository Institution:

### CABI (Centre for Agriculture and Bioscience International)

### CABI Bioscience, UK Centre (IMI)

### Bakeham Lane

### Englefield Green

### Egham

### Surrey

### TW20 9TY

Date of Deposit: 4 Jun. 2019; accepted 11 Jun. 2019

### Accession Number: IMI CC Number: 507106

Depositor: Prof. Antony Page, on behalf of The University Court of the University of Glasgow

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

A number of publications are cited above in order to more fully describe and disclose the invention and the state of the art to which the invention pertains. Full citations for these references are provided below. The entirety of each of these references is incorporated herein.
1. Eziefula A C, Brown M. Intestinal nematodes: disease burden, deworming and the potential importance of co-infection. Curr Opin Infect Dis. 2008; 21:516-22.
2. Stromberg B E, Gasbarre L C. Gastrointestinal nematode control programs with an emphasis on cattle. Vet Clin North Am Food Anim Pract. 2006; 22:543-65.
3. Fuller V L, Lilley C J, Urwin P E. Nematode resistance. New Phytol. 2008; 180:27-44.
4. Kaminsky R, Ducray P, Jung M, Clover R, Rufener L, Bouvier J, et al. A new class of anthelmintics effective against drug-resistant nematodes. Nature. 2008; 452:176-80.
5. Kotze A C, Prichard R K. Anthelmintic resistance in Haemonchus contortus: history, mechanisms and diagnosis. Adv Parasitol. 2016; 93:397-428.
6. Geerts S, Gryseels B. Drug resistance in human helminths: current situation and lessons from livestock. Clin Microbiol Rev. 2000; 13:207-22.
7. Wolstenholme A J, Fairweather I, Prichard R, von Samson-Himmelstjerna G, Sangster N C. Drug resistance in veterinary helminths. Trends Parasitol. 2004; 20:469-76.
8. Hewitson J P, Maizels R M. Vaccination against helminth parasite infections. Expert Rev Vaccines. 2014; 13:473-87.
9. Larsen M. Biological control of nematode parasites in sheep. J Anim Sci. 2006; 84 Suppl:E133-9.
10. Tian B, Yang J, Zhang K Q. Bacteria used in the biological control of plant-parasitic nematodes: populations, mechanisms of action, and future prospects. FEMS Microbiol Ecol. 2007; 61:197-213.
11. Darby C. Interactions with microbial pathogens. WormBook: the online review of C. elegans biology. 2005:1-15. doi: 10.1895/wormbook.1.21.1.
12. Riffel A, Daroit D J, Brandelli A. Nutritional regulation of protease production by the feather-degrading bacterium Chryseobacterium sp. kr6. New Biotechnol. 2011; 28:153-7.
13. Page, AP, Roberts, M, Felix, M-A, Pickard, D, Page, A, Weir W. Microbe sample from Chryseobacterium nematophagum, BioSample: SAMN09925763; Sample name: JUb129, NCBI BioSample database.
14. Félix M A, Duveau F. Population dynamics and habitat sharing of natural populations of Caenorhabditis elegans and C. briggsae. BMC Biol. 2012; 10:59.
15. Page, AP, Roberts, M, Felix, M-A, Pickard, D, Page, A, Weir W. Microbe sample from Chryseobacterium nematophagum, BioSample: SAMN09925764; Sample name: JUb275, NCBI BioSample database.
16. Herzog P, Winkler I, Wolking D, Kampfer P, Lipski A. Chryseobacterium ureilyticum sp. nov., Chryseobacterium gambrini sp. nov., Chryseobacterium pallidum sp. nov. and Chryseobacterium molle sp. nov., isolated from beer-bottling plants. Int J Systematic Evol Microbiol. 2008; 58:26-33.
17. Wu Y F, Wu Q L, Liu S J. Chryseobacterium taihuense sp. nov., isolated from a eutrophic lake, and emended descriptions of the genus Chryseobacterium, Chryseobacterium taiwanense, Chryseobacterium jejuense and Chryseobacterium indoltheticum. Int J Systematic Evol Microbiol. 2013; 63:913-9.
18. Burlinson P, Studholme D, Cambray-Young J, Heavens D, Rathjen J, Hodgkin J, et al. Pseudomonas fluorescens NZ17 repels grazing by C. elegans, a natural predator. ISME Journal. 2013; 7:1126.
19. Pradel E, Zhang Y, Pujol N, Matsuyama T, Bargmann C I, Ewbank J J. Detection and avoidance of a natural product from the pathogenic bacterium Serratia marcescens by Caenorhabditis elegans. PNAS. 2007; 104:2295-300.
20. Heustis R J, Ng H K, Brand K J, Rogers M C, Le L T, Specht C A, et al. Pharyngeal polysaccharide deacetylases affect development in the nematode C. elegans and deacetylate chitin in vitro. PloS One. 2012; 7:e40426.
21. Mango S E. The C. elegans pharynx: a model for organogenesis. In: Community TCeR, editor. WormBook: the online review of C. elegans biology 2007. (http//www.wormbook.org)
22. Page A P, Johnstone I J. The cuticle. In: Community TCeR, editor. WormBook: the online review of C. elegans biology 2007. (http://www.wormbook.org)
23. Thein M C, McCormack G, Winter A D, Johnstone I L, Shoemaker C B, Page A P. Caenorhabditis elegans exoskeleton collagen COL-19: An adult-specific marker for collagen modification and assembly, and the analysis of organismal morphology. Developmental dynamics: Amer Assoc Anat. 2003; 226:523-39.
24. Zhang Y, Foster J M, Nelson L S, Ma D, Carlow C K S. The chitin synthase genes chs-1 and chs-2 are essential for C. elegans development and responsible for chitin deposition in the eggshell and pharynx, respectively. Developmental Biol. 2005; 285:330-9.
25. McBride M J, Zhu Y. Gliding motility and Por secretion system genes are widespread among members of the phylum Bacteroidetes. J Bacteriol. 2013; 195:270-8.
26. Lauber F, Deme J C, Lea S M, Berks B C. Type 9 secretion system structures reveal a new protein transport mechanism. Nature. 2018; 564:77.
27. Chin C-S, Alexander D H, Marks P, Klammer A A, Drake J, Heiner C, et al. Nonhybrid, finished microbial genome assemblies from long-read SMRT sequencing data. Nature methods. 2013; 10:563.
28. Hunt M, De Silva N, Otto T D, Parkhill J, Keane J A, Harris S R. Circlator: automated circularization of genome assemblies using long sequencing reads. Genome Biol. 2015; 16:294.
29. Li H, Handsaker B, Wysoker A, Fennell T, Ruan J, Homer N, et al. The Sequence Alignment/Map format and SAMtools. Bioinformatics. 2009; 25:2078-9.
30. Koren S, Walenz B P, Berlin K, Miller J R, Bergman N H, Phillippy A M. Canu: scalable and accurate long-read assembly via adaptive k-mer weighting and repeat separation. Genome Res. 2017:gr. 215087.116.
31. Li H. Minimap2: pairwise alignment for nucleotide sequences. Bioinformatics. 2018; 1:7.
32. Seemann T. Prokka: rapid prokaryotic genome annotation. Bioinformatics. 2014; 30:2068-9.
33. Apweiler R, Bairoch A, Wu C H, Barker W C, Boeckmann B, Ferro S, et al. UniProt: the universal protein knowledgebase. Nucl Acid Res. 2004; 32:D115-D9.
34. Haft D H, Loftus B J, Richardson D L, Yang F, Eisen J A, Paulsen I T, et al. TIGRFAMs: a protein family resource for the functional identification of proteins. Nucl Acid Res.

2001; 29:41-3.
35. Bateman A, Coin L, Durbin R, Finn R D, Hollich V, Griffiths-Jones S, et al. The Pfam protein families database. Nucl Acid Res. 2004; 32:D138-D41.
36. Klimke W, Agarwala R, Badretdin A, Chetvernin S, Ciufo S, Fedorov B, et al. The national center for biotechnology information's protein clusters database. Nucl Acid Res. 2008; 37:D216-D23.
37. Stamatakis A. RAxML version 8: a tool for phylogenetic analysis and post-analysis of large phylogenies. Bioinformatics. 2014; 30:1312-3.
38. Train C-M, Glover N M, Gonnet G H, Altenhoff A M, Dessimoz C. Orthologous Matrix (OMA) algorithm 2.0: more robust to asymmetric evolutionary rates and more scalable hierarchical orthologous group inference. Bioinformatics. 2017; 33:i75-i82.
39. Altenhoff A M, Gil M, Gonnet G H, Dessimoz C. Inferring hierarchical orthologous groups from orthologous gene pairs. PloS One. 2013; 8:e53786.
40. Zdobnov E M, Apweiler R. InterProScan—an integration platform for the signature-recognition methods in InterPro. Bioinformatics. 2001; 17:847-8.
41. Altschul S F, Madden T L, Schaffer A A, Zhang J, Zhang Z, Miller W, et al. Gapped BLAST and PSI-BLAST: a new generation of protein database search programs. Nucl Acid Res. 1997; 25:3389-402. PM:9254694.
42. Beveridge T J. Use of the Gram stain in microbiology. Biotechnic & Histochemistry. 2001; 76:111-8.
43. Fautz E, Reichenbach H. A simple test for flexirubin-type pigments. FEMS Microbiol Letters. 1980; 8:87-91.
44. Page A P, Roberts M, Félix M-A, Pickard D, Page A, Weir W. The golden death bacillus Chryseobacterium nematophagum is a novel matrix digesting pathogen of nematodes. BMC Biology (2019) 17:10

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

1. A method of reducing parasitic nematode load in an environment, comprising treating the environment with a composition comprising Chryseobacterium nematophagum.

2. A method according to claim 1 wherein the environment is a habitat for domesticated mammals.

3. A method according to claim 2 wherein the domesticated mammals comprise livestock, equine species or domestic animals.

4.-6. (canceled)

7. A method according to claim 1 wherein the environment comprises pasture, rangeland or buildings or other structures for housing domesticated mammals.

8.-10. (canceled)

11. A method according to claim 1 comprising contacting soil or vegetation within the environment with the composition.

12. A method according to claim 1 comprising spraying the composition within the environment.

13. A method according to claim 1 wherein the parasitic nematodes are mammalian parasites.

14. A method according to claim 1 wherein the environment is a habitat for cultivated plants.

15. (canceled)

16. A method according to claim 14 wherein the cultivated plants are crop plants.

17. (canceled)

18. A method according to claim 14 comprising contacting soil or vegetation within the environment with the composition.

19. A method according to claim 14 comprising directly contacting the cultivated plants with the composition.

20. A method according to claim 14 comprising spraying the composition within the environment.

21. A method according to claim 20 comprising spraying the composition onto soil or the cultivated plants.

22. (canceled)

23. A method of reducing nematode load in an animal or reducing nematode transmission between animals comprising administering to an animal a composition comprising Chryseobacterium nematophagum.

24. A method according to claim 23 wherein the animals are domesticated mammals.

25. A method according to claim 23 comprising feeding the composition to the animal.

26. A method according to claim 23 wherein the composition comprises lyophilised C. nematophagum.

27. A method according to claim 26 wherein the lyophilised C. nematophagum is coated or encapsulated.

28. A composition comprising lyophilised C. nematophagum.

29. A composition according to claim 28 wherein the lyophilised C. nematophagum is coated or encapsulated.

30. A composition according to claim 28 in admixture with an animal feedstuff.

31. A composition according to claim 28 wherein the lyophilised C. nematophagum is coated or encapsulated.

32. (canceled)


SEQUENCE LISTING (47 sequences)

SEQ ID NO: 1 - DNA, 1427 bp, Chryseobacterium nematophagum
gatgaacgctagcgggaggcctaacacatgcaagccgagcggtatgattctttcgggaatcagagagcggcgtacgggtgcggaacacgtgtgcaacctgcctttatctgggggatagcctttcgaaaggaagattaataccccataatatattgattggcatcgattgatattgaaaactccggtggataaagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggtgcaagcctgatccagccatcccgcgtgaaggacgacggccctatgggttgtaaacttcttttgtacagggataaacctttccacgtgtgggaagctgaaggtactgtacgaataagcaccggctaactccgtgccagcagccgcggtaatacggagggtgcaagcgttatccggatttattgggtttaaagggtccgtaggcggatttgtaagtcagtggtgaaatcctacagcttaactgtagaactgccattgatactgcaagtcttgagtgtagttgaagtagctggaataagtagtgtagcggtgaaatgcatagatattacttagaacaccaattgcgaaggcaggttactaagttacaactgacgctgatggacgaaagcgtggggagcgaacaggattagataccctggtagtccacgctgtaaacgatgctaactcgtttttgggttttcggattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgacagatttagaaatagatcctccttcgggcaattttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 2 - DNA, 1427 bp, Chryseobacterium nematophagum
gatgaacgctagcgggaggcctaacacatgcaagccgagcggtatgattctttcgggaatcagagagcggcgtacgggtgcggaacacgtgtgcaacctgcctttatctgggggatagcctttcgaaaggaagattaataccccataatatattgattggcatcgattaatattgaaaactccggtggataaagatgggcacgcgcaagattagatagttggtgaggtaacggctcaccaagtccatgatctttagggggcctgagagggtgatcccccacactggtactgagacacggaccagactcctacgggaggcagcagtgaggaatattggacaatgggtgaaagcctgatccagccatcccgcgtgaaggacgacggccctatgggttgtaaacttcttttgtacagggataaacctttccacgtgtgggaagctgaaggtactgtacgaataagcaccggctaactccgtgccagcagccgcggtaatacggagggtgcaagcgttatccggatttattgggtttaaagggtccgtaggcggatttgtaagtcagtggtgaaatcctacagcttaactgtagaactgccattgatactgcaagtcttgagtgtagttgaagtagctggaataagtagtgtagcggtgaaatgcatagatattacttagaacaccaattgcgaaggcaggttactaagttacaactgacgctgatggacgaaagcgtggggagcgaacaggattagataccctggtagtccacgctgtaaacgatgctaactcgtttttgggttttcggattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgacagatttagaaatagatcctccttcgggcaattttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 3 - DNA, 1427 bp, Chryseobacterium nematophagum
gatgaacgctagcgggaggcctaacacatgcaagccgagcggtatgattctttcgggaatcagagagcggcgtacgggtgcggaacacgtgtgcaacctgcctttatctgggggatagcctttcgaaaggaagattaataccccataatatattgattggcatcgattratattgaaaactccggtggataaagatgggcacgcgcaagattagatagttggtgaggtaacggctcaccaagtccatgatctttagggggcctgagagggtgatcccccacactggtactgagacacggaccagactcctacgggaggcagcagtgaggaatattggacaatgggtgaaagcctgatccagccatcccgcgtgaaggacgacggccctatgggttgtaaacttcttttgtacagggataaacctttccacgtgtgggaagctgaaggtactgtacgaataagcaccggctaactccgtgccagcagccgcggtaatacggagggtgcaagcgttatccggatttattgggtttaaagggtccgtaggcggatttgtaagtcagtggtgaaatcctacagcttaactgtagaactgccattgatactgcaagtcttgagtgtagttgaagtagctggaataagtagtgtagcggtgaaatgcatagatattacttagaacaccaattgcgaaggcaggttactaagttacaactgacgctgatggacgaaagcgtggggagcgaacaggattagataccctggtagtccacgctgtaaacgatgctaactcgtttttgggttttcggattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgacagatttagaaatagatcctccttcgggcaattttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 4 - DNA, 1427 bp, Chryseobacterium nematophagum
gatgaacgctagcgggaggcctaacacatgcaagccgagcggtatgattctttcgggaatcagagagcggcgtacgggtgcggaacacgtgtgcaacctgcctttatctgggggatagcctttcgaaaggaagattaataccccataatatattgattggcatcgattratattgaaaactccggtggataaagatgggcacgcgcaagattagatagttggtgaggtaacggctcaccaagtcsatgatctttagggggcctgagagggtgatcccccacactggtactgagacacggaccagactcctacgggaggcagcagtgaggaatattggacaatgggtgmaagcctgatccagccatcccgcgtgaaggacgacggccctatgggttgtaaacttcttttgtacagggataaacctttccacgtgtgggaagctgaaggtactgtacgaataagcaccggctaactccgtgccagcagccgcggtaatacggagggtgcaagcgttatccggatttattgggtttaaagggtccgtaggcggatttgtaagtcagtggtgaaatcctacagcttaactgtagaactgccattgatactgcaagtcttgagtgtagttgaagtagctggaataagtagtgtagcggtgaaatgcatagatattacttagaacaccaattgcgaaggcaggttactaagttacaactgacgctgatggacgaaagcgtggggagcgaacaggattagataccctggtagtccacgctgtaaacgatgctaactcgtttttgggttttcggattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgacagatttagaaatagatcctccttcgggcaattttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 5 - DNA, 20 bp, Artificial Sequence
  primer CnemF1
tgattctttcgggaatcaga

SEQ ID NO: 6 - DNA, 22 bp, Artificial Sequence
  primer CnemR1
atatcaatcgatgccaatcaat

SEQ ID NO: 7 - DNA, 20 bp, Artificial Sequence
  Primer CnemR2
gcttcccacacgtggaaagg

SEQ ID NO: 8 - DNA, 129 bp, Artificial Sequence
  JUb129 (consensus)
tgattctttcgggaatcagagagcggcgtacgggtgcggaacacgtgtgcaacctgcctttatctgggggatagcctttcgaaaggaagattaataccccataatatattgattggcatcgattaatat

SEQ ID NO: 9 - DNA, 129 bp, Artificial Sequence; identical to SEQ ID NO: 8
  JUb275 (consensus)

SEQ ID NO: 10 - DNA, 397 bp, Artificial Sequence
  JUb129 (consensus)
tgattctttcgggaatcagagagcggcgtacgggtgcggaacacgtgtgcaacctgcctttatctgggggatagcctttcgaaaggaagattaataccccataatatattgattggcatcgattaatattgaaaactccggtggataaagatgggcacgcgcaagattagatagttggtgaggtaacggctcaccaagtccatgatctttagggggcctgagagggtgatcccccacactggtactgagacacggaccagactcctacgggaggcagcagtgaggaatattggacaatgggtgaaagcctgatccagccatcccgcgtgaaggacgacggccctatgggttgtaaacttcttttgtacagggataaacctttccacgtgtgggaagc

SEQ ID NO: 11 - DNA, 397 bp, Artificial Sequence
  JUb275 (consensus)
tgattctttcgggaatcagagagcggcgtacgggtgcggaacacgtgtgcaacctgcctttatctgggggatagcctttcgaaaggaagattaataccccataatatattgattggcatcgattgatattgaaaactccggtggataaagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggtgcaagcctgatccagccatcccgcgtgaaggacgacggccctatgggttgtaaacttcttttgtacagggataaacctttccacgtgtgggaagc

SEQ ID NO: 12 - DNA, 1427 bp, Chryseobacterium nematophagum
gatgaacgctagcgggaggcctaacacatgcaagccgagcggtatgattctttcgggaatcagagagcggcgtacgggtgcggaacacgtgtgcaacctgcctttatctgggggatagcctttcgaaaggaagattaataccccataatatattaagtggcatcacttgatattgaaaactccggtggatagagatgggcacgcgcaagattagatagttggtgaggtaacggctcaccaagtcagtgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctttccacgtgtggaaagctgaaggtactatacgaataagcaccggctaactccgtgccagcagccgcggtaatacggagggtgcaagcgttatccggatttattgggtttaaagggtccgtaggcggatgtgtaagtcagtggtgaaatctttcggcttaaccggaaaactgccattgatactgcatgtcttgagtaaggtagaagtggctggaataagtagtgtagcggtgaaatgcatagatattacttagaacaccaattgcgaaggcaggtcactatgtcttaactgacgctgatggacgaaagcgtggggagcgaacaggattagataccctggtagtccacgccgtaaacgatgctaactcgtttttgggctttcgggttcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaattgatcggtttagaaatagaccttccttcgggcaattttcaaggtgctgcatggttgtcgtcagctcgtgccgtgaggtgttaggttaagtcctgcaacgagcgcaacccctgttactagttgctaccattaagttgaggactctagtaagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 13 - DNA, 1427 bp, Chryseobacterium nematophagum
gatgaacgctagcgggaggcctaacacatgcaagccgagcggtattgtttcttcggaaatgagagagcggcgtacgggtgcggaacacgtgtgcaacctgcctttatctgggggatagcctttcgaaaggaagattaataccccataatatattgattggcatcaattaatattgaaaactccggtggatagagatgggcacgcgcaagattagatagttggtgaggtaacggctcaccaagtccatgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctttccacgtgtggaaagctgaaggtactatacgaataagcaccggctaactccgtgccagcagccgcggtaatacggagggtgcaagcgttatccggatttattgggtttaaagggtccgtaggcgggctcgtaagtcagtggtgaaatctcatagcttaactatgaaactgccattgatactgcgagccttgagtaaggtagaggtagctggaataagtagtgtagcggtgaaatgcatagatattacttagaacaccaattgcgaaggcaggttaccatgtcttaactgacgctgatggacgaaagcgtggggagcgaacaggattagataccctggtagtccacgccgtaaacgatgctaactcgtttttgggttttcggattcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaattgacaggtttagaaatagacttttcttcggacaattttcaaggtgctgcatggttgtcgtcagctcgtgccgtgaggtgttaggttaagtcctgcaacgagcgcaacccctgtcactagttgctagcattaagttgaggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 14 - DNA, 1428 bp, Chryseobacterium nematophagum
gatgaacgctagcgggaggcctaacacatgcaagccgagcggtagagattcttcgggatcttgagagcggcgtacgggtgcggaacacgtgtgcaacctgcctttatcagggagatagcctttcgaaaggaagattaatatcccataatattttgagtggcatcacttaaaattgaaagctccggcggatagagatgggcacgcgcaagattagatagttggtgaggtaacggctcaccaagtcagtgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtacagggataacagcagccgcggtaatacggagggtgcaagcgttatccggatttattgggtttaaagggtacctatttacgtgtaaatagctgaaggtactgtacgaataagcaccggctaactccgtgcccgtaggcggatctgtaagtcagtggtgaaatctcacagcttaactgtgaaactgccattgatactgcaggtcttgagtaaggtagaagtggctggaataagtagtgtagcggtgaaatgcatagatattacttagaacaccaattgcgaaggcaggtcactatgtcttaactgacgctgatggacgaaagcgtggggagcgaacaggattagataccctggtagtccacgccgtaaacgatgctaactcgtttttgggtttttaagattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgatcggtttagaaatagaccttccttcgggcaattttcaaggtgctgcatggttgtcgtcagctcgtgccgtgaggtgttaggttaagtcctgcaacgagcgcaacccctgttactagttgcyascattmagttgaggactctagtaagactgcctacgcaagtagagaggaaggtggggatgacgtcawatcatcaygscccttacgccttgggcyacacacgtaatacaatggccggtacagagggcagctacacagcgatgtgatgcmaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtytggggtacctgaagtcggtgaccgtaaaaggagctgcctagggtaaaacag

SEQ ID NO: 15 - DNA, 1427 bp, Chryseobacterium nematophagum
gatgaacgctagcgggaggcctaacacatgcaagctgagcggtagagtatcttcggagacttgagagcggcgtacgggtgcggaacacgtgtgcaacctgcctttatcagggggatagcctttcgaaaggaagattaataccccataatatatagagtggcatcactttatattgaaaactgaggtggataaagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggttagcgcctgatccagccatcccgcgtgaaggacgacggccctatgggttgtaaacttcttttgtacagggataaacctatttacgtgtaaatagctgaaggtactgtacgaataagcaccggctaactccgtgccagcagccgcggtaatacggagggtgcaagcgttatccggatttattgggtttaaagggtccgtaggcggatctgtaagtcagtgggtgaaatctcacagcttaactgtgaaactgccattgatactgcaggtcttgagtaaggtagaagtggctggaataagtagtgtagcggtgaaatgcatagatattacttagaacaccaattgcgaaggcaggtcactatgtcttaactgacgctgatggacgaaagcgtggggagcgaacaggattagataccctggtagtccacgctgtaaacgatgctaactcgtttttgggttttcggattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgatcggtttagaaatagaccttccttcgggcaattttcaaggtgctgcatggttgtcgtcagctcgtgccgtgaggtgttaggttaagtcctgcaacgagcgcaacccctgttactagttgctaccattaagttgaggactctagtaagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 16 - DNA, 1427 bp, Chryseobacterium nematophagum
gatgaacgctagcgggaggcctaacacatgcaagccgagcggtatgattctttcgggaatcagagagcggcgtacgggtgcggaacacgtgtgcaacctgcctttatctgggggatagcctttcgaaaggaagattaataccccataatatattgagtggcatcacttaatattgaaaactccggtggatagagatgggcacgcgcaagattagatagttggtggggtaacggcctaccaagtcgatgatctttagggggcctgagagggtgatcccccacactggtactgagacacggaccagactcctacgggaggcagcagtgaggaatattggacaatgggtgcaagcctgatccagccatcccgcgtgaaggacgacggccctatgggttgtaaacttcttttgtacagggataaacctagatacgtgtatctagctgaaggtactgtacgaataagcaccggctaactccgtgccagcagccgcggtaatacggagggtgcaagcgttatccggatttattgggtttaaagggtccgtaggcggatcagtaagtcagtggtgaaatctcatagcttaactatgaaactgccattgatactgctggtcttgagtaaggtagaagtagctggaataagtagtgtagcggtgaaatgcatagatattacttagaacaccaattgcgaaggcaggttactatgtcttaactgacgctgatggacgaaagcgtggggagcgaacaggattagataccctggtagtccacgctgtaaacgatgctaactcgtttttgggttttcggattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgatcggtttagaaatagaccttccttcgggcaattttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 17 - DNA, 1427 bp, Chryseobacterium nematophagum; identical to SEQ ID NO: 16

SEQ ID NO: 18 - DNA, 1427 bp, Chryseobacterium nematophagum
gatgaacgctagcgggaggcctaacacatgcaagccgagcggtattgtttcttcggaaacgagagagcggcgcacgggtgcggaacacgtgtgcaacctgcctttatctgggggatagcctttcgaaaggaagattaataccccataatattttgaatggcatcatttaaaattgaaaactccggtggatagagatgggcacgcgcaagattagatagttggtagggtaacggcctaccaagtcagtgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtacagggataaacctttccacgtgtggaaagctgaaggtactgtacgaataagcaccggctaactccgtgccagcagccgcggtaatacggagggtgcaagcgttatccggatttattgggtttaaagggtccgtaggcggatccgtaagtcagtggtgaaatctcatagcttaactatgaaactgccattgatactgcgggtcttgagtaaggtagaagtagctggaataagtagtgtagcggtgaaatgcatagatattacttagaacaccaattgcgaaggcaggttactatgtcttaactgacgctgatggacgaaagcgtggggagcgaacaggattagataccctggtagtccacgccgtaaacgatgctaactcgtttttgggctttcgggttcagagactaagcgaaagtgataagttagccacctggggagtacgaacgcaagtttgaaactcaaaggaattgacgggggcccgcacaagcggtggattatgtggtttaattcgatgatacgcgaggaaccttaccaagacttaaatgggaattgatcggtttagaaatagaccttccttcgggcaattttcaaggtgctgcatggttgtcgtcagctcgtgccgtgaggtgttaggttaagtcctgcaacgagcgcaacccctgtcactagttgccatcattcagttggggactctagtgagactgcctacgcaagtagagaggaaggtggggatgacgtcaaatcatcacggcccttacgtcttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 19 - DNA, 1427 bp, Chryseobacterium nematophagum
gatgaacgctagcgggaggcctaacacatgcaagccgagcggtagagtctcttcggggacttgagagcggcgtacgggtgcggaacacgtgtgcaacctgcctttatcagggggatagcctttcgaaaggaagattaataccccataatatattgagtggcatcacttgatattgaaaactccggtggatagagatgggcacgcgcaagattagatagttggtgaggtaacggctcaccaagtcagtgatctttagggggcctgagagggtgatcccccacactggtactgagacacggaccagactcctacgggaggcagcagtgaggaatattggacaatgggtgagagcctgatccagccatcccgcgtgaaggacgactgccctatgggttgtaaacttcttttgtacagggataaacctttctacgtgtagaaagctgaaggtactgtacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtaaggtagaagtagctggaataagtagtgtagcggtgaaatgcatagatattacttagaacaccaattgcgaaggcaggttactatgtcttaactgacgctgatggacgaaagcgtggggagcgaacaggattagataccctggtagtccacgccgtaaacgatgctaactcgtttttggggtttcggcttcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaattgacaggctcagaaatgggtttttcttcggacaattttcaaggtgctgcatggttgtcgtcagctcgtgccgtgaggtgttaggttaagtcctgcaacgagcgcaacccctgtcactagttgccatcatttagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 20 - DNA, 1427 bp, Chryseobacterium nematophagum
gatgaacgctagcgggaggcctaacacatgcaagccgagcggtagattgctttcgggcaattgagagcggcgcacgggtgcggaacacgtgtgcaacctgcctttatctgggggatagcctttcgaaaggaagattaataccccataatatgttgaatggcatcattcgacattgaaagctccggcggatagagatgggcacgggcaagattagatagttggtgaggtaacggctcaccaagtcaatgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtacagggataaacctttccacgtgtgggaagctgaaggtactgtacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtaaatttgaagtggctggaataagtagtgtagcggtgaaatgcatagatattacttagaacaccaattgcgaaggcaggtcactaagatttaactgacgctgagggacgaaagcgtggggagcgaacaggattagataccctggtagtccacgccgtaaacgatgctaactcgtttttgggttttcggattcagagactaagcgaaagtgataagttagccacctggggagtacgttcgcaagaatgaaactcaaaggaattgacgggggcccgcacaagcggtggattatgtggtttaattcgatgatacgcgaggaaccttaccaagacttaaatgggaattgacagatttagaaatagatcctccttcgggcaattttcaaggtgctgcatggttgtcgtcagctcgtgccgtgaggtgttaggttaagtcctgcaacgagcgcaacccctgtcactagttgctaccattcagttgaggactctagtgagactgcctacgcaagtagagaggaaggtggggatgacgtcaaatcatcacggcccttacgtcttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 21 - DNA, 1427 bp, Chryseobacterium nematophagum; identical to SEQ ID NO: 2

SEQ ID NO: 22 - DNA, 1427 bp, Chryseobacterium nematophagum; identical to SEQ ID NO: 1

SEQ ID NO: 23 - DNA, 1428 bp, Chryseobacterium nematophagum
gatgaacgctagcgggaggcctaacacatgcaagccgagcggtagagtctcttcggagacttgagagcggcgtacgggtgcggaacacgtgtgcaacctgcctttatctgggggatagcctttcgaaaggaagattaataccccataatattttgaatggcatcatttaaaattgaaaactccggtggatagagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggttagcgcctgatccagccatcccgcgtgaaggacgacggccctatgggttgtaaacttcttttgtacagggataaacctatctacgtgtagatagctgaaggtactgtacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgtagttgaagtagctggaataagtagtgtagcggtgaaatgcatagatattacttagaacaccaattgcgaaggcaggttactaagttacaactgacgctgatggacgaaagcgtggggagcgaacaggattagataccctggtagtccacgctgtaaacgatgcttactcgtttttgggtttttaagattcagagactaagcgaaagtgataagtaagccacctggggagtacgaacgcaagtttgaaactcaaaggaattgacgggggcccgcacaagcggtggattatgtggtttaattcgatgatacgcgaggaaccttaccaaggcttaaatgggaattgacagacgcagaaatgtgtttttcttcggacaattttcaaggtgctgcatggttgtcgtcagctcgtgccgtgaggtgttaggttaagtcctgcaacgagcgcaacccctgtcactagttgctaccattaagttgaggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 24 - DNA, 1427 bp, Chryseobacterium nematophagum
gatgaacgctagcgggaggcctaacacatgcaagctgagcggtagagatctttcgggatcttgagagcggcgtacgggtgcggaacacgtgtgcaacctgcctttatcagggggatagcctttcgaaaggaagattaataccccataatatatagagtggcatcactttatattgaaaactgaggtggataaagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggttagcgcctgatccagccatcccgcgtgaaggacgacggccctatgggttgtaaacttcttttgtacagggataaacctttccacgtgtgggaagctgaaggtactgtacgaataagcaccggctaactccgtgccagcagccgcggtaatacggagggtgcaagcgttatccggatttattgggtttaaagggtccgtaggcggatgcgtaagtcagtggtgaaatctcacagctcaactgtgaaactgccattgatactgcgtgtcttgagtgaggttgaagtagctggaataagtagtgtagcggtgaaatgcatagatattacttagaacaccaattgcgaaggcaggttactaagtctcaactgacgctgatggacgaaagcgtggggagcgaacaggattagataccctggtagtccacgctgtaaacgatgctaactcgtttttgggctttcgggttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgatcggtttagaaatagaccttccttcgggcaattttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 25 - DNA, 1427 bp, Chryseobacterium nematophagum
gatgaacgctagcgggaggcctaacacatgcaagctgagcggtagagatctttcgggatcttgagagcggcgtacgggtgcggaacacgtgtgcaacctgcctttatcagggggatagcctttcgaaaggaagattaataccccataatatatagagtggcatcactttatattgaaaactgaggtggataaagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggttagcgcctgatccagccatcccgcgtgaaggacgacggccctatgggttgtaaacttcttttgtacagggataaacctttccacgtgtgggaagctgaaggtactgtacgaataagcaccggctaactccgtgccagcagccgcggtaatacggagggtgcaagcgttatccggatttattgggtttaaagggtccgtaggcggatgcgtaagtcagtggtgaaatctcacagctcaactgtgaaactgccattgatactgcgtgtcttgagtgaggttgaagtagctggaataagtagtgtagcggtgaaatgcatagatattacttagaacaccaattgcgaaggcaggttactaagtctcaactgacgctgatggacgaaagcgtggggagcgaacaggattagataccctggtagtccacgctgtaaacgatgctaactcgtttttgggctttcgggttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgatcggtttagaaatagaccttccttcgggcaattgtcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 26 - DNA, 1427 bp, Chryseobacterium nematophagum
gatgaacgctagcgggaggcctaacacatgcaagctgagcggtagagatctttcgggatcttgagagcggcgtacgggtgcggaacacgtgtgcaacctgcctttatcagggggatagcctttcgaaaggaagattaataccccataatattttgagtggcatcacttgaaattgaaaactgaggtggataaagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggttagcgcctgatccagccatcccgcgtgaaggacgacggccctatgggttgtaaacttcttttgtacagggataaacctaccctcgtgagggtagctgaaggtactgtacgaataagcaccggctaactccgtgccagcagccgcggtaatacggagggtgcaagcgttatccggatttattgggtttaaagggtccgtaggcggatgcgtaagtcagtggtgaaatctcacagctcaactgtgaaactgccattgatactgcgtgtcttgagtgaggttgaagtagctggaataagtagtgtagcggtgaaatgcatagatattacttagaacaccaattgcgaagacaggttactaagtctcaactgacgctgatggacgaaagcgtggggagcgaacaggattagataccctggtagtccacgctgtaaacgatgctaactcgtttttggggatttatcttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgatcggtttagaaatagaccttccttcgggcaattttcaaggtgctgcatggttgtcgtcagctcgtgccgtgaggtgttaggttaagtcctgcaacgagcgcaacccctgtcactagttgccatcattcagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 27 - DNA, 1427 bp, Chryseobacterium nematophagum
gatgaacgctagcgggaggcctaacacatgcaagctgagcggtagagatctttcgggatcttgagagcggcgtacgggtgcggaacacgtgtgcaacctgcctttatcagggggatagcctttcgaaaggaagattaataccccataatattttgagtggcatcacttgaaattgaaaactgaggtggataaagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggttagcgcctgatccagccatcccgcgtgaaggacgacggccctatgggttgtaaacttcttttgtacagggataaacctaccctcgtgagggtagctgaaggtactgtacgaataagcaccggctaactccgtgccagcagccgcggtaatacggagggtgcaagcgttatccggatttattgggtttaaagggtccgtaggcggatgcgtaagtcagtggtgaaatctcacagctcaactgtgaaactgccattgatactgcgtgtcttgagtgaggttgaagtagctggaataagtagtgtagcggtgaaatgcatagatattacttagaacaccaattgcgaaggcaggttactaagtctcaactgacgctgatggacgaaagcgtggggagcgaacaggattagataccctggtagtccacgctgtaaacgatgctaactcgtttttggggatttatcttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgatcggtttagaaatagacctaccttcgggcaattttcgaggtgctgcatggttgtcgtcagctcgtgccgtgaggtgttaggttaagtcctgcaacgagcgcaacccctgtcactagttgccatcattcagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 28 - DNA, 1427 bp, Chryseobacterium nematophagum
gatgaacgctagcgggaggcctaacacatgcaagctgagcggtagagatctttcgggatcttgagagcggcgtacgggtgcggaacacgtgtgcaacctgcctttatcagggggatagcctttcgaaaggaagattaataccccataatattttgagtggcatcacttgaaattgaaaactgaggtggataaagatgggcacgcgcaagattagatagttggtgaggtaacggctcaccaagtcgatgatctttagggggcctgagagggtgatcccccacactggtactgagacacggaccagactcctacgggaggcagcagtgaggaatattggacaatgggttagcgcctgatccagccatcccgcgtgaaggacgacggccctatgggttgtaaacttcttttgtacagggataaacctaccctcgtgagggtagctgaaggtactgtacgaataagcaccggctaactccgtgccagcagccgcggtaatacggagggtgcaagcgttatccggatttattgggtttaaagggtccgtaggcggatgcgtaagtcagtggtgaaatctcacagctcaactgtgaaactgccattgatactgcgtgtcttgagtgaggttgaagtagctggaataagtagtgtagcggtgaaatgcatagatattacttagaacaccaattgcgaaggcaggttactaagtctcaactgacgctgatggacgaaagcgtggggagcgaacaggattagataccctggtagtccacgctgtaaacgatgctaactcgtttttggggatttatcttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaattgatcggtttagaaatagaccttccttcgggcaattttcaaggtgctgcatggttgtcgtcagctcgtgccgtgaggtgttaggttaagtcctgcaacgagcgcaacccctgtcactagttgccatcattcagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatagaagtctggggtacctgaagtcggtgaccgtaaaaggagctgcctagggtaaaacag

SEQ ID NO: 29 - DNA, 1427 bp, Chryseobacterium nematophagum
gatgaacgctagcgggaggcctaacacatgcaagccgagcggtaggtttccttcgggaaactgagagcggcgcacgggtgcggaacacgtgtgcaacctgcctttatcagggggatagcctttcgaaaggaagattaataccccataatattttgagtggcatcacttgaaattgaaaactccggtggataaagatgggcacgcgcaagattagatagttggtgaggtaacggctcaccaagtctacgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctttccacgtgtggaaagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagctcaactgtgaaactgccattgatactgcaggtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttggggccttggcttcagagactaagcgaaagtgataagttagccacctggggagtacggacgcaagtctgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattcagttggggactctagtgagactgcctacgcaagtagagaggaaggtggggatgacgtcaaatcatcacggcccttacgccttgggccacacacgtaatacaatggccagtacagagggcagctaccaggcgactggatgcgaatctcgaaagctggtctcagttcggattggagtctgcaactcgactctatgaagctggaatcgctagtgatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 30 - DNA, 1430 bp, Chryseobacterium nematophagum
gatgaacgctagcgggaggcctaacacatgcaagccgagcggtagagtctcttcggggacttgagagcggcgcacgggtgcggaacacgtgtgcaacctgcctttatctgggggatagcctttcgaaaggaagattaataccccataatatactggatggcatcatctggtattgaaaactccggtggatagagatgggcacgcgcaagattagatagttggtgaggtaacggctcaccaagtcagcgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctagatacgtgtatctagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttggggcgtaagcttcagagactaagcgaaagtgataagttagccacctggggagtacgaacgcaagtttgaaactcaaaggaattgacggggggcccgcacaagcggtggatttatgtggtttaattcgatgatacgcgaggaaccttaccaaggcttaaatgggaaatgacaggtttagaaatagactttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 31 - DNA, 1429 bp, Chryseobacterium nematophagum
gatgaacgctagcgggaggcctaacacatgcaagccgagcggtagagtctcttcggagacttgagagcggcgcacgggtgcggaacacgtgtgcaacctgcctttatctgggggatagcctttcgaaaggaagattaataccccataatataagaaacggcatcgttttttattgaaaactccggtggatagagatgggcacgcgcaagattagatagttggtgaggtaacggctcaccaagtctgcgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctaccctcgtgagggtagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgttgttgaagtagctggaataagtagtgtagcggtgaaatgcatagatattacttagaacaccaattgcgaaggcaggttactaagcaacaactgacgctgatggacgaaaagcgtggggagcgaacaggattagataccctggtagtccacgccgtaaacgattgctaactcgtttttgggttttcggattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaaatgaccggcttagaaataggcttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 32 - DNA, 1427 bp, Chryseobacterium nematophagum
gatgaacgctagcgggaggcctaacacatgcaagccgagcggtagagatctttcgggatcttgagagcggcgtacgggtgcggaacacgtgtgcaacctgcctttatctgggggatagcctttcgaaaggaagattaataccccataatataagaaacggcatcgttttttattgaaaactccggtggatagagatgggcacgcgcaagattagatagttggtgaggtaacggctcaccaagtctacgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctaccctcgtgagggtagctgaaggtactatacgaataagcaccggctaactccgtgccagcagccgcggtaatacggagggtgcaagcgttatccggatttattgggtttaaagggtccgtaggcggatgtgtaagtcagtggtgaaatctcacagcttaactgtgaaactgccattgatactgcatgtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttgggttttcggattcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacactgcgaagtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 33 - DNA, 1427 bp, Chryseobacterium nematophagum
gatgaacgctagcgggaggcctaacacatgcaagccgagcggtagattgctttcgggcaattgagagcggcgtacgggtgcggaacacgtgtgcaacctgcctttatctgggggatagcctttcgaaaggaagattaataccccataatataagagacggcatcgttttttattgaaaactccggtggatagagatgggcacgcgcaagattagatagttggtgaggtaacggctcaccaagtcgacgatctttagggggcctgagagggtgatcccccacactggtactgagacacggaccagactcctacgggaggcagcagtgaggaatattggacaatgggtgaaagcctgatccagccatcccgcgtgaaggacgacggccctatgggttgtaaacttcttttgtatagggataaacctttccacgagtggaaagctgaaggtactatacgaataagcaccggctaactccgtgccagcagccgcggtaatacggagggtgcaagcgttatccggatttattgggtttaaagggtccgtaggctgatttgtaagtcagtggtgaaatctcacagcttaactgtgaaactgccattgatactgcaagtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttgggatgtaagtttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaaatgacaggcttagaaataggcttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 34 - DNA, 1427 bp, Chryseobacterium nematophagum; identical to SEQ ID NO: 33

SEQ ID NO: 35 - DNA, 1427 bp, Chryseobacterium nematophagum
gatgaacgctagcgggaggcctaacacatgcaagccgagcggtagattgctttcgggcaattgagagcggcgtacgggtgcggaacacgtgtgcaacctgcctttatctgggggatagcctttcgaaaggaagattaataccccataatataagagacggcatcgttttttattgaaaactccggtggatagagatgggcacgcgcaagattagatagttggtgaggtaacggctcaccaagtcgacgatctttagggggcctgagagggtgatcccccacactggtactgagacacggaccagactcctacgggaggcagcagtgaggaatattggacaatgggtgaaagcctgatccagccatcccgcgtgaaggacgacggccctatgggttgtaaacttcttttgtatagggataaacctttccacgtgtggaaagctgaaggtactatacgaataagcaccggctaactccgtgccagcagccgcggtaatacggagggtgcaagcgttatccggatttattgggtttaaagggtccgtaggctgatttgtaagtcagtggtgaaatctcacagcttaactgtgaaactgccattgatactgcaagtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttgggatgtaagtttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaaatgacaggcttagaaataggcttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 36 - DNA, 1427 bp, Chryseobacterium nematophagum
gatgaacgctagcgggaggcctaacacatgcaagccgagcggtagattgctttcgggcaattgagagcggcgtacgggtgcggaacacgtgtgcaacctgcctttatctgggggatagcctttcgaaaggaagattaatacctcataatataagagacggcatcgttttttattgaaaactccggtggatagagatgggcacgcgcaagattagatagttggtgaggtaacggctcaccaagtcgacgatctttagggggcctgagagggtgatcccccacactggtactgagacacggaccagactcctacgggaggcagcagtgaggaatattggacaatgggtgaaagcctgatccagccatcccgcgtgaaggacgacggccctatgggttgtaaacttcttttgtatagggataaacctttccacgtgtggaaagctgaaggtactatacgaataagcaccggctaactccgtgccagcagccgcggtaatacggagggtgcaagcgttatccggatttattgggtttaaagggtccgtaggctgatttgtaagtcagtggtgaaatctcacagcttaactgtgaaactgccattgatactgcaagtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttgggatgtaagtttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaaatgacaggcttagaaataggcttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 37 - DNA, 1427 bp, Chryseobacterium nematophagum
gatgaacgctagcgggaggcctaacacatgcaagccgagcggtattgtttcttcggaaatgagagagcggcgtacgggtgcggaacacgtgtgcaacctgcctttatctgggggatagcctttcgaaaggaagattaataccccataatatattgaatggcatcattcgatattgaaaactccggtggatagagatgggcacgcgcaagattagatagttggtgaggtaacggctcaccaagtctgcgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctactctcgtgagagtagctgaaggtactatacgaataagcaccggctaactccgtgccagcagccgcggtaatacggagggtgcaagcgttatccggatttattgggtttaaagggtccgtaggcggatttgtaagtcagtggtgaaatctcacagcttaactgtgaaactgccattgatactgcaagtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttggtttttcggaatcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 38 - DNA, 1427 bp, Chryseobacterium nematophagum
gatgaacgctagcgggaggcctaacacatgcaagccgagcggtattgtttcttcggaaatgagagagcggcgtacgggtgcggaacacgtgtgcaacctgcctttatctgggggatagcctttcgaaaggaagattaataccccataatatattgaatggcatcattcgatattgaaaactccggtggatagagatgggcacgcgcaagattagatagttggtgaggtaacggctcaccaagtctacgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctactctcgtgagagtagctgaaggtactatacgaataagcaccggctaactccgtgccagcagccgcggtaatacggagggtgcaagcgttatccggatttattgggtttaaagggtccgtaggcggatgtgtaagtcagtggtgaaatctcacagcttaactgtgaaactgccattgatactgcaggtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttgggttttcggattcagagactaagcgaaagtgataagttagccacttggggagtacgttcgcaagaatgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 39 - DNA, 1427 bp, Chryseobacterium nematophagum
gatgaacgctagcgggaggcctaacacatgcaagccgagcggtatagatctttcgggatctagagagcggcgtacgggtgcggaacacgtgtgcaacctgcctttatctgggggatagcctttcgaaaggaagattaataccccataatatattgaatggcatcattcgatattgaaaactccggtggatagagatgggcacgcgcaagattagatagttggtgaggtaacggctcaccaagtctacgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctactctcgtgagagtagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttggattttcggattcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccagtacagagggcagctacacggtgacgtgatgcaaatctcgaaagctggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 40 - DNA, 1427 bp, Chryseobacterium nematophagum
gatgaacgctagcgggaggcctaacacatgcaagccgagcggtagagtctcttcggagacttgagagcggcgcacgggtgcggaacacgtgtgcaacctgcctttatctgggggatagcctttcgaaaggaagattaataccccataatatattgaatggcatcattcgatattgaaaactccggtggatagagatgggcacgcgcaagattagatagttggtgaggtaacggctcaccaagtctacgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctactctcgtgagagtagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttgggctttcgggttcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccagtacagagggcagctacacagcgatgtgatgcaaatctcgaaagctggtctcagttcggattggagtctgcaactcgactctatgaggctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacag

SEQ ID NO: 41 - DNA, 1427 bp, Chryseobacterium nematophagum
gatgaacgctagcgggaggcctaacacatgcaagccgagcggtagagatctttcgggatcttgagagcggcgtacgggtgcggaacacgtgtgcaacctgcctttatctgggggatagcctttcgaaaggaagattaataccccataatatgttggatggcatcattcgacattgaaaactccggtggatagagatgggcacgcgcaagattagatagttggtgaggtaacggctcaccaagtctgcgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctactctcgtgagagtagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttgggctttcgggttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 42 - DNA, 1427 bp, Chryseobacterium nematophagum
gatgaacgctagcgggaggcctaacacatgcaagccgagcggtagagatctttcgggatcttgagagcggcgtacgggtgcggaacacgtgtgcaacctgcctttatctgggggatagcctttcgaaagggagattaataccccataatatatttatcggcatcgatagatattgaaaactccggtggatagagatgggcacgcgcaagattagatagttggtgaggtaacggctcaccaagtctacgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctactctcgtgagagtagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgttattgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttggtatttcggtatcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 43 - DNA, 1427 bp, Chryseobacterium nematophagum
gatgaacgctagcgggaggcctaacacatgcaagccgagcggtagagatytttcgggatcttgagagcggcgtacgggtgcggaacacgtgtgcaacctgcctttatctgggggatagcctttcgaaagggagattaataccccataatatatttatcggcatcgatagatattgaaaactccggtggatagagatgggcacgcgcaagattagatagttggtgaggtaacggctcaccaagtctacgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctactctcgtgagagtagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgttattgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttggtwtttcggaatcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 44 - DNA, 1427 bp, Chryseobacterium nematophagum
gatgaacgctagcgggaggcctaacacatgcaagccgagcggtagagatctttcgggatcttgagagcggcgtacgggtgcggaacacgtgtgcaacctgcctttatcagggggatagcctttcgaaaggaagattaataccccataatatattgaatggcatcatttaatattgaaaactccggtggatagagatgggcacgcgcaagattagatagttggtgaggtaacggctcaccaagtctgcgatctttagggggcctgagagggtgatcccccacactggtactgagacacggaccagactcctacgggaggcagcagtgaggaatattggacaatgggtgcgagcctgatccagccatcccgcgtgaaggacgacggccctatgggttgtaaacttcttttgtatagggataaacctaccctcgtgagggtagctgaaggtactatacgaataagcaccggctaactccgtgccagcagccgcggtaatacggagggtgcaagcgttatccggatttattgggtttaaagggtccgtaggcggatctgtaagtcagtggtgaaatctcacagcttaactgtgaaactgccattgatactgcaggtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttggtatttcggtatcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaaatgacaggtttagaaatagacttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaaaccatggaagtctggggtacctgaagtcggtgaccgtaacaggagctgcctagggtaaaacag

SEQ ID NO: 45 - DNA, 1427 bp, Chryseobacterium nematophagum
gatgaacgctagcgggaggcctaacacatgcaagccgagcggtagagatctttcgggatcttgagagcggcgtacgggtgcggaacacgtgtgcaacctgcctttatcagggggatagcctttcgaaaggaagattaataccccataatatatcgagtggcatcacttgatattgaaaactccggtggatagagatgggcacgcgcaagattagatagttggtgaggtaacggctcaccaagtctgcgatctttagggggcctgagagggtgatcccccacactggtactgagacacggaccagactcctacgggaggcagcagtgaggaatattggacaatgggtgcgagcctgatccagccatcccgcgtgaaggatgacggccctatgggttgtaaacttcttttgtatagggataaacctactctcgtgagggtagctgaaggtactatacgaataagcaccggctaactccgtgccagcagccgcggtaatacggagggtgcaagcgttatccggatttattgggtttaaagggtccgtaggcggatgtgtaagtcagtggtgaaatctcacagcttaactgtgaaactgccattgatactgcatgtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttggtatttcggtatcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaaatgacaggcttagaaataggcttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacacagcgatgtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 46 - DNA, 1427 bp, Chryseobacterium nematophagum
gatgaacgctagcgggaggcctaacacatgcaagccgagcggtagagatctttcgggatcttgagagcggcgtacgggtgcggaacacgtgtgcaacctgcctttatctgggggatagcctttcgaaaggaagattaataccccataatatattgaatggcatcatttgatattgaaaactccggtggatagagatgggcacgcgcaagattagatagttggtgaggtaacggctcaccaagtcagcgatctttagggggcctgagagggtgatcccccacactggtactgagacacggaccagactcctacgggaggcagcagtgaggaatattggacaatgggtgagagcctgatccagccatcccgcgtgaaggacgacggccctatgggttgtaaacttcttttgtatagggataaacctaccctcgtgagggtagctgaaggtactatacgaataagcaccggctaactccgtgccagcagccgcggtaatacggagggtgcaagcgttatccggatttattgggtttaaagggtccgtaggctgatttgtaagtcagtggtgaaatctcacagcttaactgtgaaactgccattgatactgcaagtcttgagtgttgttgaagtagctggaataagtagtgtagcggtgaaatgcatagatattacttagaacaccaattgcgaaggcaggttactaagcaacaactgacgctgatggacgaaagcgtggggagcgaacaggattagataccctggtagtccacgccgtaaacgatgctaactcgtttttgggcttttgggttcagagactaagcgaaagtgataagttagccacctggggagtacgaacgcaagtttgaaactcaaaggaattgacgggggcccgcacaagcggtggattatgtggtttaattcgatgatacgcgaggaaccttaccaaggcttaaatgggaaatgacaggcttagaaataggcttttcttcggacatttttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtaatacaatggccggtacagagggcagctacactgcgaagtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaacaggagctgcctagggtaaaacag

SEQ ID NO: 47 - DNA, 1427 bp, Chryseobacterium nematophagum
gatgaacgctagcgggaggcctaacacatgcaagccgagcggtagagtatcttcggatacttgagagcggcgtacgggtgcggaacacgtgtgcaacctgcctttatctgagggatagcctttcgaaaggaagattaatacctcataacatactgattggcatcaattagtattgaaagctccggcggatagagatgggcacgcgcaagattagatagttggtgaggtaacggctcaccaagtcaatgatctttagggggcctgagagggtgatcccccacactggtactgagacacggaccagactcctacgggaggcagcagtgaggaatattggacaatgggtggaagcctgatccagccatcccgcgtgaaggacgacggccctatgggttgtaaacttcttttgtacagggataaacctactctcgtgagggtagctgaaggtactgtacgaataagcaccggctaactccgtgccagcagccgcggtaatacggagggtgcgagcgttatccggatttattgggtttaaagggtccgcaggcgggctagtaagtcagtggtgaaagcctacagcttaactgtagaactgccgttgatactgctagtcttgagtatagttgaggtagctggaatgagtagtgtagcggtgaaatgcatagatattactcagaacaccgattgcgaaggcaggttaccaagttataactgacgctgagggacgaaagcgtggggagcgaacaggattagataccctggtagtccacgccgtaaacgatgctaactcgtttttgggctttagggttcagagactaagcgaaagtgataagttagccacctggggagtacgaccgcaaggttgaaactcaaaggaattgacgggggcccgcacaagcggtggatcatgtggtttaattcgatgatacgcgaggaaccttaccaaggcttaaatgggaattgacagctgtagaaatacggttttcttcggacaattttcaaggtgctgcatggttgtcgtcagctcgtgccgtgaggtgttaggttaagtcctgcaacgagcgcaacccctgtcactagttgccatcattaagttggggactctagtgagactgcctacgcaagtagagaggaaggtggggatgacgtcaaatcatcacggcccttacgccttgggccacacacgtgatacaatggccggtacagagggcagctacactgcgaagtgatgcaaatctcgaaagccggtctcagttcggattggagtctgcaactcgactctatgaagctggaatcgctagtaatcgcgcatcagccatggcgcggtgaatacgttcccgggccttgtacacaccgcccgtcaagccatggaagctgggggtacctgaagtcggtgaccgtaacaggagctgcctagggtaaaacta